(19) United States
(12) Patent Application Publication
(10) Pub. No.: US 2021/0214758 A1
(43) Pub. Date: Jul. 15, 2021

(54) METHODS AND TOOLS FOR DETERMINING CLONAL RELATEDNESS AND PREDICTING CLONAL TRAITS

(71) Applicant: ID GENOMICS, INC., Seattle, WA (US)

(72) Inventors: Sujay CHATTOPADHYAY, Kolkata, West Bengal (IN); Veronika TCHESNOKOVA, Seattle, WA (US); Elena RECHKINA, Seattle, WA (US)

(21) Appl. No.: 17/053,727

(22) PCT Filed: May 6, 2019
(86) PCT No.: PCT/US19/30948

§ 371 Date: Nov. 6, 2020

Related U.S. Application Data
(60) Provisional application No. 62/668,042, filed on May 7, 2018.

(51) Int. Cl. C12P 19/30; C12Q 1/689; C12N 15/10

(52) U.S. Cl. CPC C12P 19/30; C12N 15/1089; C12Q 1/689

(57) ABSTRACT

The present disclosure provides methods and compositions for generating clonotypes from input nucleic acid sequences. Presently disclosed clonotype generating methods produce accurate and efficient indicators of genetic relatedness and diversity, including for example, of pathogenic organisms such as infectious bacteria. Also provided are primers, kits, and related methods for determining antibiotic susceptibility of a pathogenic organism, such as, for example, Klebsiella, and for treating an infection by one or more pathogenic Discard organisms on the basis of herein disclosed clonotypes and clonotype-generating methods.

### STATEMENT OF GOVERNMENT INTEREST

This invention was made with government support under R42AI116114-02 awarded by the National Institutes of Health. The government has certain rights in the invention.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 480361_402WO_SEQUENCE_LISTING.txt. The text file is 10.9 KB, was created on Apr. 27, 2019, and is being submitted electronically via EFS-Web.

### BACKGROUND

Increasingly, the ability to identify genetically related organisms is used to inform decision-making in a variety of contexts, including medicine and agriculture practices. For example, infectious bacteria are commonly tested for genetic relatedness to determine the likelihood that a given isolate will be susceptible or resistant to one or more antibiotics based on its predicted relatedness to bacteria with known susceptibility profiles (i.e., a shared genotype is predictive of a known, shared phenotypic trait). Determining relatedness can help avoiding potential ‘drug-bug’ mismatches, which occur in up to 25% of prescriptions (Tchesnokova et al., J. Clin. Microbiol. 51(9):2991-2999 (2013)), and misuse of up to 50% of antibiotics.

As another example, clonal relatedness and clonal frequencies of T cells (e.g., on the basis of T cell receptor gene recombination) and cancerous cells (e.g., clonal evolution as indicia of pathogenesis, immunologic escape, and resistance to therapy) are important factors in monitoring disease and choosing courses of treatment. See, e.g., Bianci and Munshi, Blood 125(20):3049-3058; see also Cha et al., Sci Transl Med. 6(238):238ra70 (2014). Identifying relatedness of clonal plants is desirable for maintaining preferred levels of homo- or heterogeneity with agricultural species; e.g., stress-resistant or herbicide-resistant strains.

However, current methods of identifying genetic relatedness typically involve costly, time-consuming techniques such as DNA sequencing, pulsed-field gel electrophoresis, microarray, and highly technical subsequent analysis. In addition, for some cell types, days of culture and preparation are also needed before sequencing and analysis can be performed. Thus, potentially crucial decisions may be delayed. Accordingly, there is a need for improved methods and tools for determining genetic relatedness.

### BRIEF DESCRIPTION OF THE DRAWINGS

FIG. 1 shows an exemplary logic process of the present disclosure for generating a clonotype from a nucleotide position library.

FIG. 2 shows a phylogenetic tree of clinical extraintestinal Klebsiella isolates from two clinical sites in Washington state (USA) based on concatenated sequences of five multi-locus sequence typing (MLST) genes (gapA, infB, mdh, phoE and rpoB) using a maximum likelihood algorithm (MEGA 7.0). Closely related branches were collapsed for visual presentation into K. oxytoca (‘Ko’) and K. pneumoniae phylogroups B2, D and F, with some sequence types (STs) remaining un-collapsed.

FIG. 3 provides a population structure analysis (spanning tree) of Klebsiella clinical isolates using eBURST v3 software. Each circle represents an individual sequence type (ST) based on sequences of 5 MLST alleles (gapA, infB, mdh, phoE, rpoB), as indicated in the figure key. Founder ST=predicted ancestor of clonal complex, CC. Co-founder ST=predicted ancestor of clonal sub-complex, SC. SLVs (single-locus variants)=unlinked STs. Links are indicated by lines connecting the STs. Each circle's size reflects its relative presence (number of isolates) in the collection of tested isolates.

FIGS. 4A-4C show the relative prevalence of antibiotic-resistant klebsiella isolates among different phylogenetically defined clonal groups. Groups are identified as phylogroups (B2 and D) within K. pneumoniae species; clonal complexes are defined within phylogroups of K. pneumoniae species or within K. oxytoca species on a level of individual clonal complexes (CC), sub-complexes (SCs), and sequence types (STs). Major (≥1.5% of all isolates) STs and CCs are shown individually; all other STs and CCc are shown combined as ‘other’. Resistance to antibiotics is presented as the percent of all resistant isolates that belong to the indicated clonal group. Antibiotics are abbreviated as follows: AMC, amoxicillin/clavulanate, CZ, cefazolin, CTR, ceftriaxone, TS, trimethoprim/sulfamethoxazole, CIP, ciprofloxacin, NIT, nitrofurantoin. Resistance levels statistically significantly higher or lower than 20% (one-tailed t-test, P<0.05) are designated with both font and background patterns as shown in the figure key, respectively; resistance levels that are statistically significantly different versus a reference clonal group (chosen as the largest group with closest to the overall pattern of resistance, marked with * on the Figure) is bold, underscored font, lower and higher resistance are as indicated (measured using multiple logistic regression for each type of clonal grouping, P<0.1). SUM-RANK was calculated as described in the Examples; N/A is stated for combined clonal groups.

FIG. 5 provides statistical calculations from 36 different 7 single-nucleotide-polymorphism (SNP) combinations (clonotypes) generated by a method of the present disclosure. The Simpson's Diversity Index and Adjusted Clonal Correlation Index values, two statistical calculations described herein, are organized as shown in the figure key.

FIG. 6 shows the results of a test wherein a 7-SNP combination for predicting a clonotype, generated using a method of the present disclosure, was compared to SNP data predicted by sequencing.

FIGS. 7A-7C show the comparison of clonotype distribution and clonotype-specific antibiotic resistance in 724 training set (‘Test’) and 728 validation set (‘Val’) Klebsiella isolates. Resistance levels to the indicated antibiotics (AMC, CZ, CS3, ESBL, TS, CIP, NIT, IMI) below 20% (i.e., less than 20% of isolates have resistance) are indicated as shown in the figure key. Statistically significant increases in clonotype prevalence or clonotype-specific resistance are indicated by bold underscored values (two-tailed Fisher's exact test).

### DETAILED DESCRIPTION

Provided herein are methods and compositions for generating a clonotype and for applying clonotype information to useful ends, such as treating disease. In certain aspects, the instant disclosure provides methods and related tools for quickly generating clonotypes that are reliable indicators of genetic relatedness and diversity. Clonotypes are generated from a library of input sequences of interest and comprise one or more genetic features selected for their ability to distinguish among and between clonally related organisms.

In other aspects, methods and compositions are provided for determining the presence or absence of a SNP from a predictive clonotype in Klebsiella. Briefly, using a clonotype-generating method as described herein, a clonotype comprised of 7 single nucleotide polymorphisms (SNPs) was interrogated using PCR and shown to predict clonal relatedness and antibiotic resistance of Klebsiella more accurately, quickly, and cost-effectively than standard multi-locus sequencing. High-fidelity primers that selectively bind and amplify the Klebsiella SNP sequences are also provided herein. Kits comprising the primers and optional additional reagents, including an optional Lookup Table that informs treatment selections based on predicted antibiotic susceptibility and Klebsiella clonotype.

In further aspects, methods are provided for determining antibiotic susceptibility of Klebsiella based on the presence or absence of the 7-SNP clonotype. In another aspect, the present disclosure provides methods for treating a Klebsiella infection in a patient.

Prior to setting forth this disclosure in more detail, it may be helpful to an understanding thereof to provide definitions of certain terms to be used herein. Additional definitions are set forth throughout this disclosure.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the term “about” means±20% of the indicated range, value, or structure, unless otherwise indicated. It should be understood that the terms “a” and “an” as used herein refer to “one or more” of the enumerated components. The use of the alternative (e.g., “or”) should be understood to mean either one, both, or any combination thereof of the alternatives. As used herein, the terms “include,” “have” and “comprise” are used synonymously, which terms and variants thereof are intended to be construed as non-limiting.

In addition, it should be understood that the individual compounds, or groups of compounds, derived from the various combinations of the structures and substituents described herein, are disclosed by the present application to the same extent as if each compound or group of compounds was set forth individually.

The term “consisting essentially of” is not equivalent to “comprising” and refers to the specified materials or steps of a claim, or to those that do not materially affect the basic characteristics of a claimed subject matter.

As used herein, the terms “phylogroup,” “phylogenetic group,” or “clonal group” are used to refer to a group of organisms having a common developmental or evolutionary history. Phylogroups may be determined on the basis of shared genetic markers, such as DNA or RNA sequences, and are sometimes depicted in a phylogenetic tree showing the evolutionary relationships between and among phylogenetic groups (e.g., similarities and differences in physical or genetic characteristics) and in relation to a common ancestor.

As used herein, the term “clonotype” refers to a set of genetic features specific to a genetically related lineage of organisms. In certain embodiments of the present disclosure, a clonotype is characterized by the presence or absence of one or more genetic markers, e.g., SNPs. In specific embodiments, a clonotype is characterized by the presence or absence of SNPs within a set of SNPs, such as a set of 5, 6, 7, 8, 9 or 10 (or more) SNPs that make up a clonotype.

As used herein, a “sequence type” (also referred to herein as a “ST”) refers to a group of organisms that share a particular combination of alleles along particular genetic loci, as determined by sequencing. In bacterial studies, sequence types are typically determined by multilocus sequence typing (MLST) comparison of alleles, wherein a bacterial isolate is characterized by DNA sequences of internal fragments of multiple housekeeping genes. Sequence typing and MLST are discussed in further detail in Larsen et al., J. Clin. Microbiol. 50(4):1355-1361 (2012), the typing techniques of which are incorporated by reference herein in their entirety. In certain embodiments, a clonotype may include organisms of one ST or from a plurality of sequence types. Loci chosen for sequence typing (or generating a clonotype) can include “housekeeping” genes that are constitutively expressed and are required for basic organismal maintenance and function; e.g., metabolism, cell growth, or the like. Chosen loci may also contain or be from coding regions for proteins specific to the type of organism of interest. For example, loci for sequence typing or for generating a clonotype for T cells can be from coding regions for T cell receptor components (e.g., V, D, and J variable region alleles), CD8 or CD4 co-receptor components, CD3 components, and so on. Loci useful for typing bacterial pathogens include coding regions for virulence factors, such as, for example, adhesins, hyaluronidases, proteases, lipases, DNases, hemolysins, endotoxins, exotoxins, iron-binding proteins, capsules, adhesion pili, flagella, lipopolysaccharides, or the like.

As used herein, a “clonal complex” refers to a collection of sequence types sharing common alleles, e.g., a collection of sequence types connected on a spanning tree (see, e.g., Teixeira et al., PLoS One 10(3):e0119315 (2015)) constructed using multi-locus sequence typing. As used herein, a “clonal sub-complex” refers to a collection of sequence types that are connected with a common founder sequence type by shared common alleles.

As used herein, “nucleic acid” or “nucleic acid molecule” or “polynucleotide” refers to any of deoxyribonucleic acid (DNA), ribonucleic acid (RNA), oligonucleotides, fragments generated, for example, by the polymerase chain reaction (PCR) or by in vitro translation, and fragments generated by any of ligation, scission, endonuclease action, or exonuclease action. In certain embodiments, the nucleic acids of the present disclosure are produced by PCR. Nucleic acids may be composed of monomers that are naturally occurring nucleotides (such as deoxyribonucleotides and ribonucleotides), analogs of naturally occurring nucleotides (e.g., α-enantiomeric forms of naturally-occurring nucleotides), or a combination of both. Modified nucleotides can have modifications in or replacement of sugar moieties, or pyrimidine or purine base moieties. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. Nucleic acid molecules can be either single stranded or double stranded. In certain embodiments of the present disclosure, nucleotides or gaps in a nucleotide sequence are named according to standard IUPAC convention, i.e., A, T, C, G, U, R (A or G), Y (C or T), S (G or C), W (A or T), K (G or T), M (A or C), B (C or G or T), D (A or G or T), H (A or C or T), V (A or C or G), N (any base), or −(gap).

The term “isolated” means that the material is removed from its original environment (e.g., the natural environment if it is naturally occurring). For example, a naturally occurring nucleic acid present in a microorganism is not isolated, but the same nucleic acid, separated from some or all of the co-existing materials in the natural system, is isolated. The term “gene” means the segment of DNA involved in producing a polypeptide chain; it includes regions preceding and following the coding region “leader and trailer” as well as intervening sequences (introns) between individual coding segments (exons). A “locus” (plural: loci) is a specific location of a gene or DNA sequence in or on a chromosome. “Alleles” are variants of a DNA sequence located at a given locus.

The term “nucleic acid amplification process” or “nucleic acid amplification reaction” refers to any process or reaction for specifically amplifying (i.e., generating one or more copies of) a target nucleic acid sequence, such as a DNA, a RNA, a or a cDNA, e.g., DNA from a pathogenic bacterium or a cell, such as a human cell. Numerous methods for amplifying nucleic acids are known, including various types of polymerase chain reaction (PCR; e.g., quantitative PCR such as QRT-PCR, ligation-mediated PCR, RT-PCR, amplified fragment length polymorphism, digital PCR, assembly PCR, touchdown PCR, nested PCR, multiplex PCR, and the like, which methods, related reagents, common reaction parameters, and common variations thereon, are known to those of ordinary skill in the art). Illustrative methods include loop-mediated isothermal amplification (LAMP) protocols that include two or three layers (depending on the number of primer pairs used) of specificity control, which can use colorimetry (double-stranded DNA dyes) or simple turbidity (Mg₂P₂O₇ precipitation) for the reaction read-out. Other isothermal amplification methods include recombinase polymerase amplification (RPA) and helicase-dependent amplification (HAD). Both methods utilize colorimetry for detection, using essentially the same instrumentation platforms as LAMP.

“Sequence identity,” as used herein, refers to the percentage of amino acid residues in one sequence that are identical with the amino acid residues in another reference polypeptide sequence after aligning the sequences and introducing gaps, if necessary, to achieve the maximum percent sequence identity, and not considering any conservative substitutions as part of the sequence identity. The percentage sequence identity values can be generated using the NCBI BLAST2.0 software as defined by Altschul et al. (1997) “Gapped BLAST and PSI-BLAST: a new generation of protein database search programs”, Nucleic Acids Res. 25:3389-3402, with the parameters set to default values.

Certain tools of statistical analysis (e.g., two-sided one-sample t-test, two-tailed Fisher's exact test) are referred to herein. In certain embodiments, modified statistical tools are referred to, which are described in detail herein.

### Methods of Generating Clonotypes

In certain aspects, the present disclosure provides methods for generating a clonotype. Clonotypes generated using the methods of this disclosure are useful for quickly and reliably identifying genetically related organisms without the need for potentially costly and time-consuming nucleic acid sequencing. Such generated clonotypes may be used, for example, to develop therapeutic regimens based on susceptibility to particular therapies.

To generate a clonotype, genetic features must be identified that are sufficiently common among individual organisms but that are also representative of the genetic diversity within a larger group of organisms; e.g., a species or a genus. More specifically, genetic features are selected for having refining power to identify closely related groups of organisms with a high degree of precision, while genetic features identified as having low or no refining power are excluded. Briefly, methods comprise: (a) generating a full binary data set from a nucleotide position library; (b) generating a reduced binary data set; (c) generating a Polymorphic Information Content (PIC) of each nucleotide position in the reduced binary data set; (d) identifying all possible pairs of nucleotide positions in the reduced binary data set; (e) generating a PIC differential; and (f) selecting non-discarded nucleotide positions to generate a clonotype. Each of these steps of the clonotype generation method is addressed in turn herein.

### Nucleotide Position Library

In some embodiments, presently disclosed methods for generating a clonotype include providing, obtaining, or constructing a nucleotide position library based on aligned and concatenated sequences from a genetic locus or from genetic loci, such as, for example, two or more alleles corresponding to a locus. As used herein, a “nucleotide position library” refers to a collection of nucleotide (e.g., purine or pyrimidine base) positions of an input nucleotide sequence or sequences. In certain embodiments, a position in a nucleotide position library corresponds to the position of the nucleotide along the input sequence; e.g., a third base in a genetic locus, such as a “G” of an “ATG” start codon of a locus is in a third nucleotide position in a library based on the locus sequence, and the “A” and “T” of the start codon are respectively in first and second positions of the library. A nucleotide position library may comprise or consist of any number of nucleotide positions (e.g., 1, 2, 3, 4, 5, 6, 7, 8, 9, or 10 nucleotide positions, or tens, or hundreds, or thousands, or tens of thousands, or hundreds of thousands, or millions of nucleotide positions, and may, in certain embodiments, comprise nucleotide positions comprising an entire genome), preferably 7 nucleotide positions. By way of illustration, Table 1 below provides an example of an initial nucleotide position library that includes 6 positions in 5 alleles.

TABLE 1
Illustrative Nucleotide Position Library

| | Position 1 | Position 2 | Position 3 | Position 4 | Position 5 | Position 6 |
|---|---|---|---|---|---|---|
| Allele 1 | A | T | G | G | A | T |
| Allele 2 | T | T | G | G | — | T |
| Allele 3 | T | T | G | T | T | A |
| Allele 4 | G | T | A | C | C | G |
| Allele 5 | C | T | C | A | G | C |

The initial nucleotide position library is then converted into a refined nucleotide position library by removing non-informative nucleotide positions. Exemplary non-informative nucleotide positions include positions having gaps or those that are monomorphic (i.e., having the same base in the same position in all of the sequences of a library), which are removed from the initial library. By way of illustration, the nucleotide position library of Table 1 includes non-informative positions at nucleotide positions 2 (monomorphic) and 5 (gap in Allele 2), so these positions are removed to produce the refined nucleotide position library shown in Table 2.

TABLE 2
Table 1 Library with Non-Informative Positions Removed

| | Position 1 | Position 3 | Position 4 | Position 6 |
|---|---|---|---|---|
| Allele 1 | A | G | G | T |
| Allele 2 | T | G | G | T |
| Allele 3 | T | G | T | A |
| Allele 4 | G | A | C | G |
| Allele 5 | C | C | A | C |

### Full Binary Data Set

Next, nucleotides at the remaining positions within the library are assigned binary data values according to their frequency of occurrence at the position. More particularly, a most-frequently occurring nucleotide at a position is assigned a first binary value, and all other nucleotide bases occurring at the position are assigned the other, different binary value, to generate a full binary data set; i.e., a binary data set that is obtained by removing gapped positions and monomorphic positions from a nucleotide position library and assigning first and second binary values to all remaining positions in the nucleotide position library as described herein. For example, for Position 1 in Table 2 above, “T” is assigned a “1”, while “A”, “G”, and “C” are each assigned a “0”. For Position 3 in Table 2, “G” is assigned a “1”, while “C”, and “A” are each assigned a “0”. For Position 4 in Table 2, “G” is assigned a “1”, while “T”, “C”, and “A” are each assigned a “0”. For Position 6 in Table 3, “T” is assigned a “1”, while “A”, “G”, and “C” are each assigned a “0”.

Assigning binary values to the refined nucleotide library shown in Table 2 generates a full binary data set, as exemplified in Table 3.

TABLE 3
Illustrative Full Binary Data Set Based on Table 2

| | Position 1 | Position 3 | Position 4 | Position 6 |
|---|---|---|---|---|
| Allele 1 | 0 | 1 | 1 | 1 |
| Allele 2 | 1 | 1 | 1 | 1 |
| Allele 3 | 1 | 1 | 0 | 0 |
| Allele 4 | 0 | 0 | 0 | 0 |
| Allele 5 | 0 | 0 | 0 | 0 |

The assigned binary values are arbitrary and may be assigned any binary value of interest, provided that the value usage is applied consistently across positions and alleles in the library. For example, although Table 3 shows the most-frequently occurring nucleotide at each position assigned a “1” and the other nucleotide bases occurring at the position assigned a “0”, the opposite binary values may be assigned; i.e., the most-frequently occurring nucleotide at each position may be assigned a “0” and the other nucleotide bases occurring at the position may be assigned a “1”.

### Reduced Binary Data Set

Next, nucleotide positions having identical (e.g., “1-0-1-0-1-0” vs. “1-0-1-0-1-0”) or reverse-identical (e.g., “1-0-1-0-1-0” vs. “0-1-0-1-0-1”) binary distribution patterns relative to other nucleotide positions provide no refining power over one another and, therefore, one nucleotide position of each pair of identical or reverse-identical nucleotide positions is removed from the library to generate a reduced binary data set, as shown in Table 4. For example, Positions 4 and 6 in Table 3 have identical distribution patterns, and, therefore, either Position 4 or Position 6 would be removed from the library to generate a reduced binary data set.

### Polymorphic Information Content (PIC)

The polymorphic information content (PIC) of each nucleotide position remaining in the reduced binary data set is calculated using a novel, modified version of the PIC calculation provided in Botstein et al., Am. J. Hum. Genet. 32:314-331 (1980), which modified version considers the subtracted square values of the frequency of each nucleotide at the position to provide information on the value of the nucleotide position as a marker of relatedness and diversity. By way of example, two nucleotide positions within a library of ten alleles have nucleotide frequencies as follows:
at position 1, 9 alleles have an “A” and 1 allele has a “G”.

PIC=1−((0.9)²+(0.1)²)=(1−(0.82))=0.18;
at position 2, 5 alleles have an “A” and the remaining 5 alleles have a G.

PIC=1−((0.5)²+(0.5)²)=(1−(0.5))=0.5.

Accordingly, a difference at position 2 separates a group of 10 alleles into two groups of 5 alleles each and is useful as a marker of diversity within the population of alleles. In contrast, a difference at position 1 separates a group of 10 alleles into one group of 9 alleles and one group of 1 allele, and is therefore less valuable as an identifier of diversity within the group of alleles. Accordingly, position 2 has higher discriminatory value than position 1.

### Polymorphic Information Content (PIC) Differential

Next, all possible pairs of nucleotide positions in the reduced binary data set are identified, and a PIC differential is generated by comparing (i) the pairwise sum of binary distribution differences between two nucleotide positions of each possible pair and (ii) the overall mean sum of binary distribution differences of all possible pairs. If the pairwise sum of binary distribution differences of (i) is smaller than the overall mean sum of the binary distribution differences of (ii), the nucleotide position with the lower PIC of the two nucleotide positions in a pair is discarded.

By way of illustration, the following calculations are made using a hypothetical data set of ten alleles (Q-Z) having three nucleotide positions (1-3) therein:

TABLE 4
Illustrative Reduced Binary Set Based on Table 3

| Allele | Position 1 | Position 2 | Position 3 |
|---|---|---|---|
| Q | 1 | 0 | 0 |
| R | 1 | 0 | 1 |
| S | 1 | 1 | 1 |
| T | 1 | 0 | 1 |
| U | 1 | 1 | 1 |
| V | 1 | 1 | 0 |
| W | 0 | 1 | 1 |
| X | 0 | 1 | 1 |
| Y | 0 | 0 | 1 |
| Z | 0 | 0 | 0 |

PIC (Position 1)=0.48
PIC (Position 2)=0.5
PIC (Position 3)=0.42
The pairwise sums of differences are as follows:
   Position 1 vs. Position 2=5
   Position 2 vs. Position 3=4
   Position 1 vs. Position 3=5
The overall mean sum of binary distribution differences of all possible pairs in the data set=((5+4+5)/3)=(14/3)=4.67

For each of the position pairs: 1 vs. 2 and 1 vs. 3, the pairwise sum of differences (5) is greater than the overall mean sum of differences in the set (4.67). For position pair: 2 vs. 3, however, the pairwise sum (4) is smaller than the overall mean sum of differences in the set (4.67). This means that the distribution of nucleotides in positions 2 and 3 is less diverse than in the data set as a whole. Thus, the nucleotide position (position 3) having the lower PIC of the position pair: 2 vs. 3 is discarded.

### Clonotype Generation

Finally, non-discarded nucleotide positions are selected based on PIC values to generate a clonotype. In certain embodiments, nucleotide positions are sorted in decreasing order of PIC values, and positions having higher PIC values are chosen for the clonotype (e.g., all nucleotide positions with PIC values above a certain predetermined threshold, or simply a predetermined number of nucleotides having the highest PIC values, such as, for example, the 5, 6, 7, 8, 9, 10, 100, etc., PIC values).

In certain embodiments, the present disclosure provides a method for generating a clonotype, wherein the method comprises:

(a) generating a full binary data set from a nucleotide position library, wherein the full binary data set comprises, for each nucleotide position in the library,
(1) an assigned first binary value to a nucleotide base that appears most frequently at the position within the library, and
(2) an assigned second binary value to all other nucleotide bases that appear at the position within the library, wherein the first and second assigned binary values are different;

wherein the nucleotide position library comprises an aligned, concatenated nucleic acid sequence set obtained from one or more loci in a genome, in which (i) nucleotide positions with a gap and (ii) nucleotide positions that are monomorphic in the sequence set are discarded;

(b) generating a reduced binary data set, comprising discarding from the full binary data set one nucleotide position from each pair of nucleotide positions having identical or reverse-identical binary distribution patterns;

(c) generating a Polymorphic Information Content (PIC) of each nucleotide position in the reduced binary data set, wherein

PIC=[1−Σ(frequency of the assigned first binary value at the position)²+(frequency of assigned second binary value at the position)²)];

(d) identifying all possible pairs of nucleotide positions in the reduced binary data set;

(e) generating a PIC differential, wherein the PIC differential comprises:
(i) a pairwise sum of binary distribution differences between two nucleotide positions of a pair for each of the possible pairs of the nucleotide positions in the reduced binary data set,
(ii) an overall mean sum of binary distribution differences in the reduced binary data set based on all possible pairs of the nucleotide positions in the reduced binary data set,

wherein the nucleotide position with the lower PIC of the two nucleotide positions in a pair is discarded when the pairwise sum of binary distribution differences of (i) is smaller than the overall mean sum of the binary distribution differences of (ii); and

(f) selecting non-discarded nucleotide positions to generate a clonotype.

In further embodiments, a method for generating a clonotype further comprises, following (e)(ii) and prior to (f), ordering the non-discarded nucleotide positions according to PIC value; e.g., PIC (position 1)>PIC (position 2)>PIC (position 4)>PIC (position 4)>PIC (position 5). Without wishing to be bound by theory, nucleotide positions having a higher PIC value have higher diversity than nucleotide positions having a lower PIC value, and may provide for a more informative clonotype.

In any of the aforementioned embodiments, a nucleotide position library can comprise nucleic acid sequences from one or more one allele of the one or more locus. Allelic sequences for various loci of various organisms are available online, including at, for example, the National Center for Biotechnology Information (NCBI) online (ncbi.hlm.nih.gov).

In any of the aforementioned embodiments, a nucleotide position library of the present disclosure can comprise nucleic acid sequences from a bacterium; a human cell, which in some embodiments comprises a T cell (see, e.g., Thor Straten et al., J. Transl. Med. 2(1):11 (2004)); a tumor; a non-human animal; or a plant.

In certain embodiments, a nucleotide position library comprises sequences a bacterium, such as, for example, an infectious bacterium. In particular embodiments, a nucleotide position library comprises sequences from: Acinetobacter baumannii; Actinomyces israelii; Actinomyces gerencseriae; Anaplasma species; Ancylostoma braziliense; Angiostrongylus; Anisakis; Arcanobacterium haemolyticum; Junin virus; Ascaris lumbricoides; Aspergillus species; an Astroviridae family member; Anaplasma phagocytophilum; Actinomycetoma sp.; Babesia sp.; Bacillus anthracis, Bacillus cereus; Bacillus sp.; Bacteroides sp.; Balantidium coli; Bartonella; Batrachochytrium dendrabatidis; Baylisascaris species; Blastocystis sp.; Blastomyces dermatitidis; Bartonella bacilliformis; Bartonella henselae; Borrelia burgdorferi; Borrelia hermsii; Borrelia recurrentis; Borrelia garinii; Borrelia afzeliil; Bordetella pertussis; Brucella sp.; Brevibacterium sp.; Burkholderia mallei; Burkholderia pseudomallei; Burkholderia cepacia; Campylobacter sp.; Candida sp.; Capillaria philippinensis; Capillaria hepatica; Capillaria aerophile, Chlamydia trachomatis; Chlamydophila pneumoniae; Chlamydophila psittaci; Citrobacter freundii; Citrobacter koserii; Citrobacter sedlakii; Citrobacter sp.; Clonorchis sinensis; Corynebacterium diphtheria; Corynebacterium sp; Clostridium botulinum; Clostridium difficile; Clostridium tetani; Clostridium perfringens; Clostridium sp; Coxiella burnetii; Cryptococcus neoformans; Cryptosporidium sp.; Cyclospora cayetanensis; Escherichia coli; Escherichia coli O157:H7, Escherichia coli O111; Escherichia coli O104.H4; Ehrlichia ewingii; Ehrlichia chaffeensis; Ehrlichia sp.; Echinococcus sp.; Enterococcus faecalis; Enterococcus faecium; Enterococcus sp.; Entamoeba histolytica; Enterobacter aerogenes; Enterobacter cloacae; Fusobacterium sp.; Fonsecaea pedrosoi; Francisella tularensis; Geotrichum candidum; Haemophilus ducreyi; Haemophilus influenza; Helicobacter pylori; Klebsiella pneumoniae; Klebsiella oxytoca; Klebsiella granulomatis; Klebsiella variicola; Klebsiella sp.; Kingella kingae; Kluyvera ascorbate, Legionella pneumophila; Leptospira sp.; Listeriamonocytogenes; Mycobacterium tuberculosis; Mycobacterium ulcerans; Mycobacterium leprae; Mycobacterium lepromatosis; Mycoplasma pneumoniae; Moraxella sp.; Morganella morganii; Neisseria gonorrhoeae; Neisseria meningitides; Nocardia asteroids; Piedraia hortae; Pantoea agglomerans; Pseudomonas aeruginosa; Pseudomonas sp.; Proteus mirabilis; Proteus sp.; Pasteurella sp.; Prevotella sp.; Propionibacterium propionicus; Rickettsia rickettsia; Rickettsia prowazekii; Rickettsia typhi; Rickettsia akari; Raoultella ornithinolytica; Raoultella planticola; Raoultella sp.; Streptococcus pneumoniae; Streptococcus pyogenes; Streptococcus agalactiae; Streptococcus sp.; Salmonella enterica subsp. Enterica; serovar typhi; Salmonella sp.; Shigella sp.; Staphylococcus aureus; Staphylococcus saprophyticus; Staphylococcus epidermidis; Staphylococcus haemolyticus Staphylococcus sp.; Serratia marcensens; Serratia liquefaciens; Serratia grimesii; Serratia maltophilia; Trypanosoma brucei; Trichosporon beigelii; Ureaplasma urealyticum; Vibrio cholera; Vibrio vulnificus; Vibrio parahaemolyticus; Yersinia pestis; Yersinia enterocolitica, or Yersinia pseudotuberculosis.

Nucleotide sequences of various organisms, such as infectious bacteria, and including sequences from bacterial strains and alleles of bacterial genes, can be readily found using, for example, the ENTREZ genome browsing tool (ncbi.nlm.nih.gov) or the PATRIC genome browser (e.g., v3.5.11; patricbrc.org/view/DataType/Genomes).

In some embodiments, a nucleotide position library comprises sequences from one or more loci that is associated with a predetermined sequence type (ST) or multi-locus sequence typing (MLST) scheme (see, e.g., Larsen et al., J. Clin. Microbiol. 50(4):1355-1361 (2012), the typing techniques of which are incorporated by reference herein in their entirety; see also the online MLST databases at pubmlst.org/databases.shtml, which organisms, databases, sequence types, isolates, and published references are incorporated herein by reference. However, a predetermined sequence type or MLST scheme is not required, and a nucleotide position library of the present disclosure may be constructed using whole genome sequence or sequences from any part thereof, and in the absence of a known ST or MLST scheme.

In certain embodiments, generating a clonotype comprises selecting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, or more non-discarded nucleotide positions, each having a PIC value above a pre-determined threshold PIC value or otherwise having a desired PIC value. In particular embodiments, selecting the one or more nucleotide positions comprises selecting 7 nucleotide positions (e.g., for testing via PCR using an 8-well PCR tube strip or plate, with a control reaction in the 8^{th} well).

In further embodiments, a clonotype-generating method of the instant disclosure further comprises testing the generated clonotype on a sample comprising nucleic acids from the organism or cell type of interest (i.e., the organism or cell type from which the nucleic acid sequences comprising the nucleotide position library were obtained), wherein the organism or cell type of interest is of one or more predetermined sequence type, wherein the testing comprises:

(a) performing an amplification reaction on the sample using forward and reverse primers for the clonotype nucleotide positions; and

(b) comparing results from the amplification reaction with the one or more predetermined sequence type.

In some embodiments, the nucleic acid amplification reaction comprises a polymerase chain reaction (PCR), such as, for example, a quantitative polymerase chain reaction (qPCR).

### Klebsiella-Specific Primers, Lookup Tables, and Kits

In certain embodiments, primers are provided for use in nucleic acid amplification processes of this disclosure; e.g., to detect the presence or absence of SNPs in Klebsiella, or to test a generated clonotype on a sample. As referred to herein, primers (also referred to herein as forward polynucleotides, forward oligonucleotides, reverse polynucleotides, or reverse oligonucleotides) are typically short oligonucleotides (e.g., ranging from about 10 to about 35 bases) that serve as the starting material for a nucleic acid amplification process. Primers typically include at least one region of sequence that is complementary to a target sequence to be amplified, and in some cases are perfectly complementary to a target sequence over their full length. In certain embodiments, a primer contains one or more introduced SNP relative to the complementary sequence of the target sequence, such that the primer sequence is not perfectly complementary to the nucleic acid sequence to be amplified in at least one nucleotide position. Without wishing to be bound by theory, certain such introduced SNPs allow for improved fidelity in target-specific nucleic acid amplification reactions relative to reference a primer sequence that does not contain the introduced one or more SNPs, and can thereby reduce or eliminate false positive results. In some embodiments, a primer comprises a deletion (i.e., one or more missing nucleotide, which may comprise contiguous missing nucleotides) relative to the template sequence to be amplified.

In some embodiments, primers of the present disclosure are designed to hybridize with sequences that contain genetic features (e.g., SNPs) identified according to the presently disclosed clonotyping methods (e.g., Klebsiella SNPs as disclosed herein) and are suitable for use in any nucleic acid amplification process. Although the results of a nucleic acid amplification process of the present disclosure can be evaluated by turbidity or using UV-light (e.g., SYBR-Green dye), other known methods and instruments may be used to visualize a result of a nucleic acid amplification process, such as, for example, the ESE-Quant Tube Scanner (Qiagen, Inc), the Genie IITM (Pro-Lab Diagnostics, Inc.), the Rotor-Gene Q instrument for RT-PCR, or the like.

In certain embodiments, a primer comprises a forward primer sequence or a reverse primer sequence according to any one of SEQ ID NOs: 1-48, 60, or 61. In further embodiments, a primer pair is provided, wherein the primers are capable of selectively amplifying a target sequence comprising a phoE54 SNP, a rpoB130 SNP, a infB279 SNP, a mdh315 SNP, a phoE336 SNP, a phoE354 SNP, or a mdh429 SNP. In some embodiments, primer pairs are provided that comprise one or more of the following primer pairs:

(i) a phoE54 forward primer comprising or consisting of the nucleic acid sequence of SEQ ID NO:4 and a phoE54 reverse primer comprising or consisting of the nucleic acid sequence of SEQ ID NO:8 or SEQ ID NO:11,

(ii) a rpoB130 forward primer comprising or consisting of the nucleic acid sequence of SEQ ID NO:20 or SEQ ID NO:19 and a rpoB130 reverse primer comprising or consisting of the nucleic acid sequence of SEQ ID NO:25 or SEQ ID NO:60,

(iii) a infB279 forward primer comprising or consisting of the nucleic acid sequence of SEQ ID NO:29 and a infB279 reverse primer comprising or consisting of the nucleic acid sequence of SEQ ID NO:33 or SEQ ID NO:35,

(iv) a mdh315 forward primer comprising or consisting of the nucleic acid sequence of SEQ ID NO:41 or SEQ ID NO:49 and a mdh315 reverse primer comprising or consisting of the nucleic acid sequence of SEQ ID NO:42,

(v) a phoE336 forward primer comprising or consisting of the nucleic acid sequence of SEQ ID NO:10 and a phoE336 reverse primer comprising or consisting of the nucleic acid sequence of SEQ ID NO:11,

(vi) a phoE354 forward primer comprising the nucleic acid sequence of SEQ ID NO:16 and a phoE354 reverse primer comprising the nucleic acid sequence of SEQ ID NO:11, and

(vii) a mdh429 forward primer comprising or consisting of the nucleic acid sequence of any one of SEQ ID NOs:45, 41, or 49, and a mdh429 reverse primer comprising or consisting of the nucleic acid sequence of SEQ ID NO:48.

As used herein, “phoE54” refers to a SNP that is an “A” nucleotide at position 54 of the gene encoding outer membrane pore protein E (phoE) in Klebsiella. UniProt entries exist for phoE of a number of Klebsiella species, strains, and subspecies, including K. pnemoniae, K. oxytoca, K. michiganensis, K. quasipneumoniae, K. aerogenes, K. LTGPAF-6F, K. OBRC7, K. RIT-PI-d, K. variicola, and related strains and subspecies.

As used herein, “rpoB130” refers to a SNP that is a “G” nucleotide at position 130 of the gene encoding DNA-directed RNA polymerase subunit beta in Klebsiella. UniProt entries exist for rpoB of a number of Klebsiella species, strains, and subspecies, including K. pnemoniae, K. oxytoca, K. michiganensis, K. quasipneumoniae, K. aerogenes, K. LTGPAF-6F, K. OBRC7, K. RIT-PI-d, K. variicola, and related strains and subspecies.

As used herein, “infB279” refers to a SNP that is a “T” nucleotide at position 279 of the gene encoding translation initiation factor IF-2 (infB) in Klebsiella. UniProt entries exist for infB of a number of Klebsiella species, strains, and subspecies, including K. pnemoniae, K. oxytoca, K. michiganensis, K. quasipneumoniae, K. aerogenes, K. LTGPAF-6F, K. OBRC7, K. RIT-PI-d, K. variicola, and related strains and subspecies.

As used herein, “mdh315” refers to a SNP that is a “C” nucleotide at position 279 of the gene encoding methanol dehydrogenase (mdh) in Klebsiella. UniProt entries exist for mdh of a number of Klebsiella species, strains, and subspecies, including K. pnemoniae, K. oxytoca, K. michiganensis, K. quasipneumoniae, K. aerogenes, K. LTGPAF-6F, K. OBRC7, K. RIT-PI-d, K. variicola, and related strains and subspecies.

As used herein, “phoE336” refers to a SNP that is a “G” nucleotide at position 336 of the Klebsiella phoE gene. As used herein, “phoE354” refers to a SNP that is a “C” nucleotide at position 354 of the Klebsiella phoE gene.

As used herein, “mdh429” refers to a SNP that is a “C” nucleotide at position 429 of the Klebsiella mdh gene.

In certain embodiments, the primer pairs comprise:

(i) a phoE54 forward primer comprising or consisting of the nucleic acid sequence of SEQ ID NO:4 and a phoE54 reverse primer comprising or consisting of the nucleic acid sequence of SEQ ID NO:8 or SEQ ID NO:11,

(ii) a rpoB130 forward primer comprising or consisting of the nucleic acid sequence of SEQ ID NO:20 or SEQ ID NO:19 and a rpoB130 reverse primer comprising or consisting of the nucleic acid sequence of SEQ ID NO:25 or SEQ ID NO:60,

(iii) a infB279 forward primer comprising or consisting of the nucleic acid sequence of SEQ ID NO:29 and a infB279 reverse primer comprising or consisting of the nucleic acid sequence of SEQ ID NO:33 or SEQ ID NO:35,

(iv) a mdh315 forward primer comprising or consisting of the nucleic acid sequence of SEQ ID NO:41 or SEQ ID NO:49 and a mdh315 reverse primer comprising or consisting of the nucleic acid sequence of SEQ ID NO:42,

(v) a phoE336 forward primer comprising or consisting of the nucleic acid sequence of SEQ ID NO:10 and a phoE336 reverse primer comprising or consisting of the nucleic acid sequence of SEQ ID NO:11,

(vi) a phoE354 forward primer comprising the nucleic acid sequence of SEQ ID NO:16 and a phoE354 reverse primer comprising the nucleic acid sequence of SEQ ID NO:11, and

(vii) a mdh429 forward primer comprising or consisting of the nucleic acid sequence of any one of SEQ ID NOs:45, 41, or 49, and a mdh429 reverse primer comprising or consisting of the nucleic acid sequence of SEQ ID NO:48.

In certain embodiments, a Klebsiella SNP is located within a sequence that is hybridized by, or is amplified by an amplification reaction containing, a forward primer or a reverse primer of the present disclosure, as shown in Table 11.

In certain embodiments, a primer of the instant disclosure can comprise a naturally occurring nucleotide, a modified nucleotide, or both. “Naturally occurring nucleotides” includes deoxyribonucleotides and ribonucleotides. The term “modified nucleotides” includes nucleotides with modified or substituted sugar groups or the like (e.g., modified with bromouridine, arabinoside, or 2′3′-dideoxyribose). The term “oligonucleotide linkages” includes oligonucleotide linkages such as phosphorothioate, phosphorodithioate, phosphoroselenoate, phosphorodiselenoate, phosphoroanilothioate, phoshoraniladate, phosphoroamidate, or the like. See, e.g., LaPlanche et al., 1986, Nucl. Acids Res., 14:9081; Stec et al., 1984, J. Am. Chem. Soc., 106:6077; Stein et al., 1988, Nucl. Acids Res., 16:3209; Zon et al., 1991, Anti-Cancer Drug Design, 6:539; Zon et al., 1991, OLIGONUCLEOTIDES AND ANALOGUES: A PRACTICAL APPROACH, pp. 87-108 (F. Eckstein, Ed.), Oxford University Press, Oxford England; Stec et al., U.S. Pat. No. 5,151,510; Uhlmann and Peyman, 1990, Chemical Reviews, 90:543, the disclosures of which are hereby incorporated by reference for any purpose. A primer or a nucleotide of this disclosure can, in some embodiments, include a detectable label to enable detection of the primer or hybridization thereof.

In some embodiments, primers are provided that are capable of hybridizing under moderate to high stringency conditions to a target sequence as provided herein, or a fragment thereof, or a complementary sequence thereof. By way of illustration, suitable moderately stringent conditions for testing the hybridization of a polynucleotide as provided herein with other polynucleotides include prewashing in a solution of 5×SSC, 0.5% SDS, 1.0 mM EDTA (pH 8.0); hybridizing at 50° C.-60° C., 5×SSC, overnight; followed by washing twice at 65° C. for 20 minutes with each of 2×, 0.5× and 0.2×SSC containing 0.1% SDS. The stringency of hybridization can be readily manipulated, such as by altering the salt content of the hybridization solution and/or the temperature at which the hybridization is performed. For example, suitable highly stringent hybridization conditions include those described above, with the exception that the temperature of hybridization is increased, e.g., to 60° C.-65° C. or 65° C. 70° C. As described herein, primers that are “specific for” a particular target or template sequence can hybridize with the target or template sequence at a temperature of at least about 57° C. or above.

Also provided herein are kits that comprise one or more of the herein disclosed primers or primer pairs and optional additional components. In certain embodiments, a kit is provided that comprises:

(a) forward primer and reverse primer pairs for at least seven Klebsiella single nucleotide polymorphisms (SNPs), wherein the SNPs comprise phoE54, rpoB130, infB279, mdh315, phoE336, phoE354, and mdh429, and wherein the primer pairs comprise one or more of the following primer pairs:

(i) a phoE54 forward primer comprising or consisting of the nucleic acid sequence of SEQ ID NO:4 and a phoE54 reverse primer comprising or consisting of the nucleic acid sequence of SEQ ID NO:8 or SEQ ID NO:11,

(ii) a rpoB130 forward primer comprising or consisting of the nucleic acid sequence of SEQ ID NO:20 or SEQ ID NO:19 and a rpoB130 reverse primer comprising or consisting of the nucleic acid sequence of SEQ ID NO:25 or SEQ ID NO:60,

(iii) a infB279 forward primer comprising or consisting of the nucleic acid sequence of SEQ ID NO:29 and a infB279 reverse primer comprising or consisting of the nucleic acid sequence of SEQ ID NO:33 or SEQ ID NO:35,

(iv) a mdh315 forward primer comprising or consisting of the nucleic acid sequence of SEQ ID NO:41 or SEQ ID NO:49 and a mdh315 reverse primer comprising or consisting of the nucleic acid sequence of SEQ ID NO:42,

(v) a phoE336 forward primer comprising or consisting of the nucleic acid sequence of SEQ ID NO:10 and a phoE336 reverse primer comprising or consisting of the nucleic acid sequence of SEQ ID NO:11,

(vi) a phoE354 forward primer comprising the nucleic acid sequence of SEQ ID NO:16 and a phoE354 reverse primer comprising the nucleic acid sequence of SEQ ID NO:11, and

(vii) a mdh429 forward primer comprising or consisting of the nucleic acid sequence of any one of SEQ ID NOs:45, 41, or 49, and a mdh429 reverse primer comprising or consisting of the nucleic acid sequence of SEQ ID NO:48;

(b) optional additional reagents for performing a nucleic acid amplification reaction (e.g., one or more of a polymerase, such as a Taq polymerase, a buffer for the polymerase, a polymerase reaction cofactor such as MgCl₂, Mg2⁺, K⁺, a nucleotide mix, a nucleic acid stain such as SYBR Green 1, dimethylsulfoxide (DMSO), sterile water, formamide, bovine serum albumin (BSA), and Betaine);

(c) an optional Lookup Table; and

(d) an optional instruction for identifying a Klebsiella clonotype and determining the Klebsiella susceptibility to one or more antibiotics.

In further embodiments, the Lookup Table is Lookup Table 1. The information contained in Lookup Table 1 below was obtained from urine specimens from patients with Klebsiella infections from several clinics within different regions of the United States. CT=clonotypes determined using a 7-SNP combination: phoE54; rpoB130; infB279; mdh315; phoE336; phoE354; and mdh429. Other abbreviations: ampicillin (AMP), amoxicillin/clavulanate (AMC), CS1 (first generation cephalosporins), CS3 (third generation cephalosporins), trimethoprim/sulfamethoxazole (TS), ciprofloxacin (CIP), nitrofurantoin (NIT), imipenem (IMI), fosfomycin (FOS), tetracycline (TET), and ceftazidime vs ceftazidime/clavulanate to determine production of extended-spectrum beta-lactamases (ESBLs). “Y” and “N” indications of allowance or non-recommendation, respectively, are based on a 20% resistance threshold to the indicated antibiotic; Y indicates that more than 80% of the tested isolates within the indicated clonotype are susceptible to the indicated antibiotic, and N indicates that less than 80% of the isolates are susceptible to the antibiotic.

TABLE 5
Lookup Table 1.

| | ANTIBIOTIC ALLOWED (Y) OR NOT RECOMMENDED (N) (susceptibility, %) | | | | | | | | | | |
|---|---|---|---|---|---|---|---|---|---|---|---|
| CT | AMP | AMC | CS1 | CS3 | TS | CIP | NIT | IMI | FOS | TET | ESBL? |
| Any | N (6) | Y (87) | Y (86) | Y (91) | N (80) | Y (91) | N (59) | Y (99) | N (41) | Y (82) | Y (95) |
| Klebsiella spp. | | | | | | | | | | | |
| 000 | N (34) | Y (92) | N (76) | Y (93) | Y (87) | Y (92) | Y(91) | Y (100) | N (63) | Y (93) | Y (98) |
| 211 | N (2) | Y (90) | Y (96) | Y (97) | Y (91) | Y(95) | Y(81) | Y (100) | N (43) | Y (88) | Y (99) |
| 131 | N (0) | N (69) | N (75) | Y (82) | N (72) | Y (90) | N (56) | Y (98) | N (36) | N (52) | Y (90) |
| 051 | N (0) | Y (90) | Y (91) | Y (93) | Y (85) | Y (97) | N (56) | Y (100) | N (41) | Y (85) | Y (93) |
| 151 | N (2) | N (78) | Y (85) | Y (88) | N (65) | Y (84) | N (41) | Y (100) | N (43) | N (75) | Y (91) |
| 331 | N (1) | Y (86) | Y (86) | Y (88) | N (76) | Y (92) | N (53) | Y (100) | N (32) | N (75) | Y (94) |
| 150 | N (0) | Y (87) | Y (82) | Y (85) | N (73) | Y (82) | N (42) | Y (99) | N (42) | Y (80) | Y (87) |
| 130 | N (2) | Y (98) | Y (98) | Y (98) | Y (92) | Y (100) | N (70) | Y (100) | N (29) | Y (88) | Y (100) |
| 550 | N (0) | Y (86) | Y (90) | Y (94) | N (79) | Y (94) | N (19) | Y (98) | N (33) | N (75) | Y (98) |
| 351 | N (0) | Y (92) | Y (94) | Y (94) | Y (84) | Y (94) | N (59) | Y (100) | N (35) | Y (86) | Y (97) |
| 251 | N (0) | Y (93) | Y (93) | Y (95) | Y (80) | Y (93) | N (57) | Y (100) | N (40) | Y (83) | Y (97) |
| 731 | N (0) | Y (98) | Y (95) | Y (96) | Y (82) | Y (93) | N (67) | Y (100) | N (43) | Y (85) | Y (96) |
| 201 | N (6) | Y (89) | Y (94) | Y (96) | N (79) | Y (96) | N (66) | Y (100) | N (26) | Y (83) | Y (98) |
| 730 | N (0) | N (60) | N (62) | N (64) | N (45) | N (48) | N (36) | Y (98) | N (21) | N (79) | N (64) |
| 171 | N (0) | Y (92) | Y (92) | Y (94) | Y (92) | Y (97) | N (28) | Y (97) | N (22) | Y (81) | Y (94) |
| 350 | N (0) | Y (83) | Y (86) | Y (86) | N (74) | Y (89) | N (54) | Y (100) | N (44) | Y (83) | Y (89) |
| 650 | N (0) | Y(91) | Y (94) | Y (94) | Y (83) | Y (94) | N (63) | Y (100) | N (37) | Y (83) | Y (94) |
| 200 | N (9) | N (69) | N (66) | Y (83) | N (77) | Y (89) | Y (97) | Y (100) | N (43) | Y (83) | Y (86) |
| 530 | N (0) | Y (93) | Y (93) | Y (93) | N (45) | Y (93) | N (31) | Y (100) | N (14) | N (75) | Y (96) |
| 551 | N (0) | N (50) | N (54) | N (61) | N (52) | NO (54) | N (18) | NO (75) | N (36) | Y (93) | Y (93) |
| 651 | N (4) | Y (92) | Y (100) | Y (100) | Y (85) | Y (92) | N (35) | Y (100) | N (24) | Y (92) | Y (100) |
| 301 | N (0) | Y (96) | Y (100) | Y (100) | N (76) | Y (100) | N (64) | Y (100) | N (24) | Y (88) | Y (100) |
| 271 | N (0) | Y (96) | Y (96) | Y (96) | N (75) | Y (96) | N (50) | Y (100) | N (38) | N (79) | Y (96) |
| 170 | N (4) | Y (96) | Y (87) | Y (91) | Y (83) | Y(91) | N (39) | Y (96) | N (39) | Y (91) | Y (96) |
| 210 | N (0) | N (71) | N (76) | Y (86) | Y (95) | Y(95) | N (57) | Y (100) | N (45) | N (76) | Y (90) |
| 050 | N (0) | Y (90) | Y (86) | Y (90) | Y (86) | Y (90) | N (33) | Y (100) | N (29) | N (76) | Y (90) |
| 750 | N (0) | Y(95) | Y (100) | Y (100) | N (65) | Y(95) | N (50) | Y (100) | N (60) | Y (95) | Y (100) |
| 311 | N (0) | Y (80) | Y (80) | Y (85) | N (65) | Y (80) | N (60) | Y (100) | N (35) | Y (80) | Y (85) |
| 250 | N (0) | Y (100) | Y (100) | Y (100) | Y (80) | Y (100) | N (50) | Y (100) | N (55) | Y (85) | Y (100) |
| 330 | N (0) | Y(95) | Y (100) | Y (100) | N (55) | Y (90) | N (15) | Y (100) | N (40) | Y (80) | Y (100) |
| 310 | N (0) | Y (80) | Y (87) | Y (93) | Y (100) | Y (100) | N (47) | Y (100) | N (33) | Y (93) | Y (93) |
| 771 | N (0) | Y (100) | Y (100) | Y (100) | Y (86) | Y (100) | N (57) | Y (100) | N (36) | Y (100) | Y (100) |
| 011 | N (0) | Y(91) | Y (91) | Y (91) | Y (91) | Y (100) | Y (82) | Y (100) | N (9) | N (73) | Y (100) |
| 111 | N (0) | Y (90) | Y (90) | Y (90) | Y (90) | Y (100) | N (30) | Y (100) | N (30) | Y (90) | Y (100) |
| 571 | N (0) | Y (100) | Y (100) | Y (100) | Y (100) | Y (100) | N (50) | Y (100) | N (25) | Y (100) | Y (100) |
| 100 | N (0) | Y (86) | N (43) | N (71) | Y (100) | Y (100) | Y(86) | Y (100) | N (71) | Y (100) | Y (100) |
| 450 | N (0) | Y (100) | Y (100) | Y (100) | Y (83) | Y (83) | N (50) | Y (100) | N (33) | Y (83) | Y (100) |
| 751 | N (17) | Y (100) | Y (100) | Y (100) | Y (100) | Y (100) | N (33) | Y (100) | N (17) | Y (100) | Y (100) |
| 611 | N (0) | Y (100) | Y (100) | Y (100) | Y (100) | Y (100) | N (50) | Y (100) | N (33) | N (67) | Y (100) |
| 531 | N (0) | Y (83) | Y (83) | Y (100) | N (67) | Y (100) | N (33) | Y (100) | N (33) | Y (100) | Y (100) |
| 071 | N (0) | Y (80) | Y (80) | Y (80) | N (60) | Y (80) | N (60) | Y (100) | Y (80) | Y (80) | Y (80) |
| 570 | N (0) | N (75) | Y (100) | Y (100) | N (75) | N (75) | N (25) | Y (100) | N (50) | N (75) | Y (100) |
| 370 | N (0) | Y (100) | Y (100) | Y (100) | N (75) | Y (100) | N (25) | Y (100) | N (25) | N (75) | Y (100) |
| 371 | N (0) | N (75) | N (67) | N (75) | Y (100) | Y (100) | N (25) | Y (100) | N (50) | Y (100) | Y (100) |
| 040 | N (25) | N (75) | N (75) | N (75) | N (25) | N (50) | N (50) | NO (75) | N (25) | N (50) | Y (100) |
| 451 | N (0) | Y (100) | Y (100) | Y (100) | Y (100) | Y (100) | N (67) | Y (100) | N (33) | Y (100) | Y (100) |
| 031 | N (0) | N (67) | Y (100) | Y (100) | N (67) | Y (100) | Y (100) | NO (67) | N (33) | N (67) | Y (100) |
| 710 | N (0) | Y (100) | Y (100) | Y (100) | N (67) | Y (100) | N (67) | Y (100) | N (33) | N (67) | Y (100) |
| 010 | N (0) | Y (100) | Y (100) | Y (100) | Y (100) | Y (100) | N (33) | Y (100) | N (33) | N (67) | Y (100) |
| 670 | N (0) | Y (100) | Y (100) | Y (100) | Y (100) | Y (100) | N (0) | Y (100) | N (0) | Y (100) | Y (100) |
| 510 | N (0) | Y (100) | Y (100) | Y (100) | Y (100) | Y (100) | N (0) | Y (100) | N (50) | Y (100) | Y (100) |
| 411 | N (0) | Y (100) | Y (100) | Y (100) | Y (100) | Y (100) | N (50) | Y (100) | N (50) | Y (100) | Y (100) |
| 231 | N (0) | Y (100) | Y (100) | Y (100) | Y (100) | Y (100) | N (50) | Y (100) | Y (100) | Y (100) | Y (100) |
| 230 | N (0) | Y (100) | Y (100) | Y (100) | Y (100) | Y (100) | N (0) | Y (100) | Y (100) | N (0) | Y (100) |
| 001 | N (0) | Y (100) | Y (100) | Y (100) | Y (100) | Y (100) | N (0) | Y (100) | N (0) | N (0) | Y (100) |
| 110 | N (0) | Y (100) | Y (100) | Y (100) | Y (100) | Y (100) | N (0) | Y (100) | N (0) | Y (100) | Y (100) |
| 300 | N (0) | Y (100) | Y (100) | Y (100) | N (0) | N (0) | N (0) | Y (100) | N (0) | N (0) | Y (100) |
| 160 | N (0) | Y (100) | Y (100) | Y (100) | N (0) | Y (100) | N (0) | Y (100) | N (0) | Y (100) | Y (100) |
| 240 | N (0) | N (0) | N (0) | N (0) | Y (100) | Y (100) | Y (100) | Y (100) | Y (100) | Y (100) | Y (100) |
| 141 | N (0) | Y (100) | Y (100) | Y (100) | Y (100) | Y (100) | N (0) | Y (100) | N (0) | Y (100) | Y (100) |
| 511 | N (0) | Y (100) | Y (100) | Y (100) | N (0) | Y (100) | Y (100) | Y (100) | Y (100) | Y (100) | Y (100) |
| 241 | N (0) | Y (100) | Y (100) | Y (100) | Y (100) | Y (100) | N (0) | Y (100) | N (0) | Y (100) | Y (100) |
| 630 | N (0) | Y (100) | Y (100) | Y (100) | Y (100) | Y (100) | N (0) | Y (100) | N (0) | Y (100) | Y (100) |

In further embodiments, a Lookup Table is constructed as described in U.S. Patent Publication No. US 2016/0251702; e.g., assigning the probability that an isolate belonging to a particular clonotype will be sensitive or resistant to different antibiotics on a scale from 0 to 100, with 0 being completely resistant and 100 being completely sensitive. If 90-100% bacteria that belong to the particular clonotype are sensitive to particular antibiotic, the respective cell in the Lookup Table is colored green, and this antibiotic is recommended to be used for treatment; pale green indicates 80-90% sensitivity level, and treatment is allowed too. Yellow (75-80%) and orange (70-75%) indicate that treatment is still allowed but with caution, and switching to a different antibiotic is recommended. Red indicates that more than 30% of bacteria are resistant to this antibiotic, and the latter should be rejected as a choice for treatment. Of course, it will be understood that a Lookup Table can be designed or adjusted to reflect differences in relevant characteristics of the clonotyped organism (e.g., resistance profiles of Klebsiella isolates (for example, such differences as may be seen from Klebsiella isolates from other collection points and/or other collection periods), antigen specificity of T cells, tumor susceptibility to chemotherapies and immunotherapies, etc.), and may include information about other or additional antibiotics or other reagents, and may provide still other information.

In particular embodiments, the primer pairs of a kit comprise:

(i) a phoE54 forward primer comprising or consisting of the nucleic acid sequence of SEQ ID NO:4 and a phoE54 reverse primer comprising or consisting of the nucleic acid sequence of SEQ ID NO:8 or SEQ ID NO:11,

(ii) a rpoB130 forward primer comprising or consisting of the nucleic acid sequence of SEQ ID NO:20 or SEQ ID NO:19 and a rpoB130 reverse primer comprising or consisting of the nucleic acid sequence of SEQ ID NO:25 or SEQ ID NO:60,

(iii) a infB279 forward primer comprising or consisting of the nucleic acid sequence of SEQ ID NO:29 and a infB279 reverse primer comprising or consisting of the nucleic acid sequence of SEQ ID NO:33 or SEQ ID NO:35,

(iv) a mdh315 forward primer comprising or consisting of the nucleic acid sequence of SEQ ID NO:41 or SEQ ID NO:49 and a mdh315 reverse primer comprising or consisting of the nucleic acid sequence of SEQ ID NO:42,

(v) a phoE336 forward primer comprising or consisting of the nucleic acid sequence of SEQ ID NO:10 and a phoE336 reverse primer comprising or consisting of the nucleic acid sequence of SEQ ID NO:11,

(vi) a phoE354 forward primer comprising the nucleic acid sequence of SEQ ID NO:16 and a phoE354 reverse primer comprising the nucleic acid sequence of SEQ ID NO:11, and

(vii) a mdh429 forward primer comprising or consisting of the nucleic acid sequence of any one of SEQ ID NOs:45 or 41 or 49, and a mdh429 reverse primer comprising or consisting of the nucleic acid sequence of SEQ ID NO:48.

In still further embodiments, at least two of the primer pairs selected from (a)(i)-(a)(vii) are mixed in a single container.

### Detecting SNPs in Klebsiella

In another aspect, the present disclosure provides methods for determining the presence or absence of a single nucleotide polymorphism (SNP) in Klebsiella, wherein a method comprises performing a nucleic acid amplification process on DNA isolated from Klebsiella obtained from a patient sample, wherein the nucleic acid amplification process comprises use of forward and reverse primer pairs specific for at least seven different Klebsiella single nucleotide polymorphisms (SNPs), wherein the at least seven different SNPs comprise phoE54, rpoB130, infB279, mdh315, phoE336, phoE354, and mdh429, and determining the presence or absence of one or more of the phoE54, rpoB130, infB279, mdh315, phoE336, phoE354, and mdh429 SNPs.

Briefly, the presently disclosed SNPs were identified using a herein disclosed clonotype generating method as useful indicators of Klebsiella clonality and antibiotic resistance. In certain embodiments, the SNP-specific primer pairs comprise a forward and a reverse primer selected from SEQ ID NOs:1-48 and 60-61 that, in an amplification reaction, can specifically amplify a SNP-containing region of interest.

In certain embodiments, the primer pairs comprise one or more of the following primer pairs:

(i) a phoE54 forward primer comprising or consisting of the nucleic acid sequence of SEQ ID NO:4 and a phoE54 reverse primer comprising or consisting of the nucleic acid sequence of SEQ ID NO:8 or SEQ ID NO:11,

(ii) a rpoB130 forward primer comprising or consisting of the nucleic acid sequence of SEQ ID NO:20 or SEQ ID NO:19 and a rpoB130 reverse primer comprising or consisting of the nucleic acid sequence of SEQ ID NO:25 or SEQ ID NO:60,

(iii) a infB279 forward primer comprising or consisting of the nucleic acid sequence of SEQ ID NO:29 and a infB279 reverse primer comprising or consisting of the nucleic acid sequence of SEQ ID NO:33 or SEQ ID NO:35,

(iv) a mdh315 forward primer comprising or consisting of the nucleic acid sequence of SEQ ID NO:41 or SEQ ID NO:49 and a mdh315 reverse primer comprising or consisting of the nucleic acid sequence of SEQ ID NO:42,

(v) a phoE336 forward primer comprising or consisting of the nucleic acid sequence of SEQ ID NO:10 and a phoE336 reverse primer comprising or consisting of the nucleic acid sequence of SEQ ID NO:11,

(vi) a phoE354 forward primer comprising the nucleic acid sequence of SEQ ID NO:16 and a phoE354 reverse primer comprising the nucleic acid sequence of SEQ ID NO:11, and

(vii) a mdh429 forward primer comprising or consisting of the nucleic acid sequence of any one of SEQ ID NOs:45 or 41 or 49, and a mdh429 reverse primer comprising or consisting of the nucleic acid sequence of SEQ ID NO:48.

In certain embodiments, the primer pairs comprise:

(i) a phoE54 forward primer comprising or consisting of the nucleic acid sequence of SEQ ID NO:4 and a phoE54 reverse primer comprising or consisting of the nucleic acid sequence of SEQ ID NO:8 or SEQ ID NO:11,

(ii) a rpoB130 forward primer comprising or consisting of the nucleic acid sequence of SEQ ID NO:20 or SEQ ID NO:19 and a rpoB130 reverse primer comprising or consisting of the nucleic acid sequence of SEQ ID NO:25 or SEQ ID NO:60,

(iii) a infB279 forward primer comprising or consisting of the nucleic acid sequence of SEQ ID NO:29 and a infB279 reverse primer comprising or consisting of the nucleic acid sequence of SEQ ID NO:33 or SEQ ID NO:35,

(iv) a mdh315 forward primer comprising or consisting of the nucleic acid sequence of SEQ ID NO:41 or SEQ ID NO:49 and a mdh315 reverse primer comprising or consisting of the nucleic acid sequence of SEQ ID NO:42,

(v) a phoE336 forward primer comprising or consisting of the nucleic acid sequence of SEQ ID NO:10 and a phoE336 reverse primer comprising or consisting of the nucleic acid sequence of SEQ ID NO:11,

(vi) a phoE354 forward primer comprising the nucleic acid sequence of SEQ ID NO:16 and a phoE354 reverse primer comprising the nucleic acid sequence of SEQ ID NO:11, and

(vii) a mdh429 forward primer comprising or consisting of the nucleic acid sequence of any one of SEQ ID NOs:45 or 41 or 49, and a mdh429 reverse primer comprising or consisting of the nucleic acid sequence of SEQ ID NO:48.

### Determining Antibiotic Susceptibility

In yet another aspect, the present disclosure provides methods for determining antibiotic susceptibility of a pathogenic organism, such as, e.g., an infectious bacteria, wherein the methods comprise (a) amplifying fragments from a genome (e.g., polynucleotide fragments from a genome) of the pathogenic organism using primer pairs specific for one or more SNPs, wherein the one or more SNPs constitute a clonotype, (b) detecting the presence or absence of the one or more SNPs to identify the clonotype, and (c) comparing the clonotype to a Lookup Table that correlates one or more clonotype of the pathogenic organism with an antibiotic susceptibility profile (e.g., whether known clonotypes of the pathogenic organism are known to be susceptible to one or more antibiotic or antibiotic class as described herein. In certain embodiments, the one or more SNPs that constitute a clonotype are identified using a clonotype-generating method of the present disclosure. In some embodiments, a method comprises (a) amplifying fragments comprising one or more SNPs of the present disclosure; e.g., phoE54, rpoB130, infB279, mdh315, phoE336, phoE354, and mdh429, in a pathogenic organism, (b) detecting the presence or absence of the one or more SNPs to identify the clonotype, and (c) comparing the clonotype to a Lookup Table.

For example, in certain embodiments, a method for determining antibiotic susceptibility of Klebsiella comprises:

(a) amplifying polynucleotide fragments from a Klebsiella genome using forward and reverse primer pairs specific for at least seven different Klebsiella single nucleotide polymorphisms (SNPs), wherein the at least seven different SNPs comprise phoE54, rpoB130, infB279, mdh315, phoE336, phoE354, and mdh429, and wherein the primer pairs comprise a phoE54-specific primer pair, a rpoB130-specific primer pair, a infB279-specific primer pair, a mdh315-specific primer pair, a phoE336-specific primer pair, a phoE354-specific primer pair, and a mdh429-specific primer pair, according to the SNP-specific forward and reverse primers of SEQ ID NOs: 1-48;

(b) detecting the presence or absence of one or more of the at least seven SNPs in the Klebsiella genome to identify the Klebsiella clonotype; and

(c) comparing the Klebsiella clonotype to a Lookup Table to determine the Klebsiella susceptibility to one or more antibiotics.

In certain embodiments, the primer pairs comprise one or more of the following primer pairs:

(i) a phoE54 forward primer comprising or consisting of the nucleic acid sequence of SEQ ID NO:4 and a phoE54 reverse primer comprising or consisting of the nucleic acid sequence of SEQ ID NO:8 or SEQ ID NO:11,

(ii) a rpoB130 forward primer comprising or consisting of the nucleic acid sequence of SEQ ID NO:20 or SEQ ID NO:19 and a rpoB130 reverse primer comprising or consisting of the nucleic acid sequence of SEQ ID NO:25 or SEQ ID NO:60,

(iii) a infB279 forward primer comprising or consisting of the nucleic acid sequence of SEQ ID NO:29 and a infB279 reverse primer comprising or consisting of the nucleic acid sequence of SEQ ID NO:33 or SEQ ID NO:35,

(iv) a mdh315 forward primer comprising or consisting of the nucleic acid sequence of SEQ ID NO:41 or SEQ ID NO:49 and a mdh315 reverse primer comprising or consisting of the nucleic acid sequence of SEQ ID NO:42,

(v) a phoE336 forward primer comprising or consisting of the nucleic acid sequence of SEQ ID NO:10 and a phoE336 reverse primer comprising or consisting of the nucleic acid sequence of SEQ ID NO:11,

(vi) a phoE354 forward primer comprising the nucleic acid sequence of SEQ ID NO:16 and a phoE354 reverse primer comprising the nucleic acid sequence of SEQ ID NO:11, and

(vii) a mdh429 forward primer comprising or consisting of the nucleic acid sequence of any one of SEQ ID NOs:45 or 41 or 49, and a mdh429 reverse primer comprising or consisting of the nucleic acid sequence of SEQ ID NO:48.

In certain embodiments, the primer pairs comprise:

(i) a phoE54 forward primer comprising or consisting of the nucleic acid sequence of SEQ ID NO:4 and a phoE54 reverse primer comprising or consisting of the nucleic acid sequence of SEQ ID NO:8 or SEQ ID NO:11,

(ii) a rpoB130 forward primer comprising or consisting of the nucleic acid sequence of SEQ ID NO:20 or SEQ ID NO:19 and a rpoB130 reverse primer comprising or consisting of the nucleic acid sequence of SEQ ID NO:25 or SEQ ID NO:60,

(iii) a infB279 forward primer comprising or consisting of the nucleic acid sequence of SEQ ID NO:29 and a infB279 reverse primer comprising or consisting of the nucleic acid sequence of SEQ ID NO:33 or SEQ ID NO:35,

(iv) a mdh315 forward primer comprising or consisting of the nucleic acid sequence of SEQ ID NO:41 or SEQ ID NO:45 and a mdh315 reverse primer comprising or consisting of the nucleic acid sequence of SEQ ID NO:42,

(v) a phoE336 forward primer comprising or consisting of the nucleic acid sequence of SEQ ID NO:10 and a phoE336 reverse primer comprising or consisting of the nucleic acid sequence of SEQ ID NO:11,

(vi) a phoE354 forward primer comprising the nucleic acid sequence of SEQ ID NO:16 and a phoE354 reverse primer comprising the nucleic acid sequence of SEQ ID NO:11, and

(vii) a mdh429 forward primer comprising or consisting of the nucleic acid sequence of any one of SEQ ID NOs:45 or 41 or 49, and a mdh429 reverse primer comprising or consisting of the nucleic acid sequence of SEQ ID NO:48.

In any of the herein disclosed kits or methods, a reference primer pair specific for mdh and comprising the nucleotide sequences according to SEQ ID NO:49 (forward primer) and 55 (reverse primer) may be used. Other reference primers specific for mdh that may be used in any of the herein disclosed kits or methods comprise or consist of a nucleotide sequence according to SEQ ID NOS:50-54 or 56-59.

In any of the herein disclosed methods of determining an antibiotic susceptibility, an antibiotic comprises a penicillin (e.g., ampicillin (AMP), mezlocillin, piperacillin, ticarcillin, methicillin, oxacillin, and the like), amoxicillin/clavulanate (A/C), a first-generation cephalosporin, a second-generation cephalosporin (e.g., cefaclor, cefamandole, cefonicid, ceforanide, cefuroxime, and the like), a third generation cephalosporin, a fourth generation cephalosporin (e.g., ceflidine, cefepime, cefluprenam, cefozopan, cefpirome, cefquinome, and the like), trimethoprim/sulfamethoxazole (T/S), a fluorquinolone, nitrofurantoin (NIT), a tetracycline (TET)(e.g., doxycycline, minocycline, oxytetracycline, tetracycline, and the like), a macrolide (e.g., azithromycin, clarithromycin, dirithromycin, erythromycin, roxithromycin, troleandomycin, and the like), an aminoglycoside (e.g., amikacin, gentamicin, kanamycin, neomycin, streptomycin, tobramycin, and the like), imipenem (IMI), ceftazidime/clavulanate, or any combination thereof.

In some embodiments, a first-generation cephalosporin comprises cefadoxil, cephradine, cefazolin, cephalexin, cefacetrile, cefadroxyl, cephaloglycin, cephalonium, cephaloridine, cephalothin, cephapirin, cephatrizine, cefazaflur, cefazedone, cefradine, cefroxadine, ceftezole, or any combination thereof. In particular embodiments, a first-generation cephalosporin comprises cefazolin.

In certain embodiments, a third-generation cephalosporin comprises cefcapene, cefdaloxime, cefdinir, cefditoren, cefetamet, cefixime, cefmenoxime, cefodizime, cefotaxime, cefovecin, cefpimizole, cefpodoxime, cefteram, ceftamere, ceftibuten, ceftiofur, ceftiolene, ceftizoxime, ceftriaxone, cefopperazone, ceftazidime, oxacephem, latomoxef, or any combination thereof. In particular embodiments, a third-generation cephalosporin comprises ceftriaxone (CTR).

In some embodiments, a fluorquinolone comprises flumequine, oxolinic acid, rosoxacin, ciprofloxacin, fleroxacin, lomefloxacin, nadifloxacin, norflocaxin, ofloxacin, pefloxacin, rufloxacin, balofloxacin, grepafloxacin, levofloxacin, pazufloxacin, sparfloxacin, temafloxacin, cinafloxacin, gatifloxacin, moxiflocaxin, sitafloxacin, prulifloxacin, trovafloxacin, clinafloxacin, or any combination thereof. In particular embodiments, a fluorquinolone comprises ciproflaxin (CIP).

### Treating Infections

Also provided herein are methods for treating an infection in a patient, wherein a method comprises administering to a patient in need thereof an effective amount of one or more antibiotic, wherein a pathogenic organism infecting the patient is known to be susceptible to the one or more administered antibiotics as determined using a method or a kit according to the present disclosure.

In certain embodiments, a pathogenic organism infecting a patient comprises Klebsiella. Briefly, Klebsiella bacteria are typically found in human intestines and feces, where they do not cause disease. Infections most commonly occur in hospital or healthcare settings and typically enter via the respiratory tract (e.g., via ventilators), urinary tract, bloodstream (e.g., via a contaminated intravenous catheter), surgical sites, or wounds, and can cause pneumonia, meningitis, sepsis, fever, chills, rash, light-headedness, and other conditions and symptoms. Standard treatment includes antibiotics and combinations thereof, though some Klebsiella are resistant to most antibiotics, including, in some instances, carbapenems.

As understood by a person skilled in the medical art, the terms, “treat” and “treatment,” refer to medical management of a disease, disorder, or condition of a subject (i.e., patient, host, who may be a human or non-human animal) (see, e.g., Stedman's Medical Dictionary). In general, an appropriate dose and treatment regimen provide one or more antibiotic in an amount sufficient to provide therapeutic or prophylactic benefit. Therapeutic or prophylactic benefit resulting from therapeutic treatment or prophylactic or preventative methods include, for example an improved clinical outcome, wherein the object is to prevent or retard or otherwise reduce (e.g., decrease in a statistically significant manner relative to an untreated control) an undesired physiological change or disorder, or to prevent, retard or otherwise reduce the expansion or severity of such a disease or disorder. Beneficial or desired clinical results from treating a subject include abatement, lessening, or alleviation of symptoms that result from or are associated the disease or disorder to be treated; decreased occurrence of symptoms; improved quality of life; longer disease-free status (i.e., decreasing the likelihood or the propensity that a subject will present symptoms on the basis of which a diagnosis of a disease is made); diminishment of extent of disease; stabilized (i.e., not worsening) state of disease; delay or slowing of disease progression; amelioration or palliation of the disease state; and remission (whether partial or total), whether detectable or undetectable; or overall survival.

“Treatment” can also mean prolonging survival when compared to expected survival if a subject were not receiving treatment. Subjects in need of the methods and compositions described herein include those who already have the disease or disorder, as well as subjects prone to have or at risk of developing the disease or disorder. Subjects in need of prophylactic treatment include subjects in whom the disease, condition, or disorder is to be prevented (i.e., decreasing the likelihood of occurrence or recurrence of the disease or disorder). The clinical benefit provided by the compositions (and preparations comprising the compositions) and methods described herein can be evaluated by design and execution of in vitro assays, preclinical studies, and clinical studies in subjects to whom administration of the compositions is intended to benefit (e.g., by slowed or reversed rates of infection spread, by lower bacterial counts in patient samples, reduction in severity of symptoms, and so on).

A “therapeutically effective amount” or “effective amount” of a composition (e.g., antibiotic or combination of antibiotics) of this disclosure refers to that amount of compound sufficient to result in amelioration of one or more symptoms of the disease being treated in a statistically significant manner. When referring to an individual active ingredient, administered alone, a therapeutically effective dose refers to the effects of that ingredient alone. When referring to a combination, an effective dose refers to the combined amounts of active ingredients or combined adjunctive active ingredient with a composition (such as, for example, an antibiotic) that results in a therapeutic effect, whether administered serially or simultaneously. An appropriate dose, suitable duration, and frequency of administration of the compositions will be determined by such factors as the age, size, gender, and condition of the patient; the type and severity of the disease, condition, or disorder; the particular form of the active ingredient; and the method of administration.

In certain embodiments, a method of treating an infection (e.g., a Klebsiella infection) in a patient comprises administering one or more antibiotics selected from a penicillin (e.g., ampicillin (AMP), mezlocillin, piperacillin, ticarcillin, methicillin, oxacillin, and the like), amoxicillin/clavulanate (A/C), a first-generation cephalosporin, a second-generation cephalosporin (e.g., cefaclor, cefamandole, cefonicid, ceforanide, cefuroxime, and the like), a third generation cephalosporin, a fourth generation cephalosporin (e.g., ceflidine, cefepime, cefluprenam, cefozopan, cefpirome, cefquinome, and the like), trimethoprim/sulfamethoxazole (T/S), a fluorquinolone, nitrofurantoin (NIT), a tetracycline (TET)(e.g., doxycycline, minocycline, oxytetracycline, tetracycline, and the like), a macrolide (e.g., azithromycin, clarithromycin, dirithromycin, erythromycin, roxithromycin, troleandomycin, and the like), an aminoglycoside (e.g., amikacin, gentamicin, kanamycin, neomycin, streptomycin, tobramycin, and the like), imipenem (IMI), ceftazidime/clavulanate, or any combination thereof.

In some embodiments, a first-generation cephalosporin comprises cefadoxil, cephradine, cefazolin, cephalexin, cefacetrile, cefadroxyl, cephaloglycin, cephalonium, cephaloridine, cephalothin, cephapirin, cephatrizine, cefazaflur, cefazedone, cefradine, cefroxadine, ceftezole, or any combination thereof. In particular embodiments, a first-generation cephalosporin comprises cefazolin.

In certain embodiments, a third-generation cephalosporin comprises cefcapene, cefdaloxime, cefdinir, cefditoren, cefetamet, cefixime, cefmenoxime, cefodizime, cefotaxime, cefovecin, cefpimizole, cefpodoxime, cefteram, ceftamere, ceftibuten, ceftiofur, ceftiolene, ceftizoxime, ceftriaxone, cefopperazone, ceftazidime, oxacephem, latomoxef, or any combination thereof. In particular embodiments, a third-generation cephalosporin comprises ceftriaxone (CTR).

In some embodiments, a fluorquinolone comprises flumequine, oxolinic acid, rosoxacin, ciprofloxacin, fleroxacin, lomefloxacin, nadifloxacin, norflocaxin, ofloxacin, pefloxacin, rufloxacin, balofloxacin, grepafloxacin, levofloxacin, pazufloxacin, sparfloxacin, temafloxacin, cinafloxacin, gatifloxacin, moxiflocaxin, sitafloxacin, prulifloxacin, trovafloxacin, clinafloxacin, or any combination thereof. In particular embodiments, a fluorquinolone comprises ciproflaxin (CIP).

In any of the presently disclosed methods of detecting the presence or absence of a SNP, or of determining antibiotic susceptibility of a pathogenic organism (e.g., Klebsiella), or of treating an infection in a patient, the pathogenic organism may be from a patient sample (e.g., a wound swab, an abscess aspirate, a fecal swab, urine, blood, saliva, sputum, a nasal swab, a tracheal swab, or a skin swipe), an invasive medical instrument (e.g., a bronchoscope, a breathing tube, a catheter, a surgical instrument, or the like), or a patient-accessible surface in a healthcare or elder care setting (e.g., a wall, a floor, a curtain, a bedsheet, a pillow, a doorknob, etc., in a hospital, an urgent care clinic, an ambulance, a physician's office, a nursing home, or the like).

In some embodiments, the pathogenic organism is from a patient sample selected from the group consisting of a wound swab, an abscess aspirate, a fecal swab, urine, blood, saliva, sputum, a nasal swab, a tracheal swab, and a skin swipe.

In further embodiments, the patient sample is or was previously fractionated to separate bacterial components from non-bacterial nucleic acids, ureas, and solids, prior to performing the nucleic acid amplification process. In still further embodiments, fractionated bacteria from a patient sample are lysed or were lysed prior to performing the amplification process or step.

### EXAMPLES

### Example 1

### Generation of a Klebsiella Clonotype

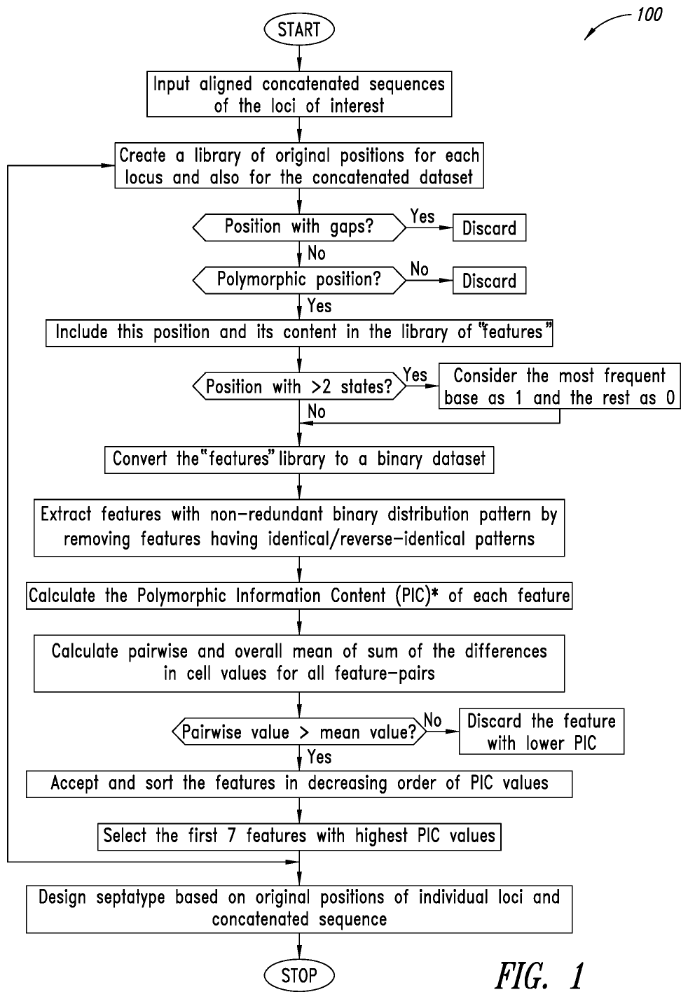

A clonotype-generating process was designed to identify features of genetic relatedness based on input sequences. A flow chart showing the steps of an exemplary clonotype-generating process is provided in FIG. 1.

To directly compare the resolution provided by a generated-clonotype with the resolution provided by standard multi-locus sequence typing (MLST) approach, known sequence types, or “STs” (combinations of alleles of five housekeeping Klebsiella loci for both K. pneumoniae and oxytoca species; bigsdb.pasteur.fr/klebsiella/klebsiella.html) were obtained without a priori knowledge as to whether the STs could be used to determine genetic relatedness of the Klebsiella clinical isolates. The alleles were from the gapA, infB, mdh, phoE and rpoB loci. The allele sequences were then concatenated and aligned to create a nucleotide position library. Nucleotide positions with a gap in one or more of the input allele sequences or that were monomorphic across the input allele sequences were discarded from the library, such that the resulting library consisted of non-gapped, polymorphic nucleotide positions.

Next, a full binary data set was created by assigning binary values to each position remaining within the library; specifically, a first binary value was assigned to the nucleotide base that appeared most frequently at the position and a second, different, binary value was assigned to all other nucleotide bases that appeared at the position within the library.

A reduced binary data set was then generated by discarding from the full binary data set one nucleotide position from each pair of nucleotide positions that had identical or reverse-identical binary distribution patterns; in other words, if a first nucleotide position shared an identical (e.g., “1-0-1-0-1-0” vs. “1-0-1-0-1-0”) or reverse-identical (e.g., “1-0-1-0-1-0” vs. “0-1-0-1-0-1”) binary distribution with a second nucleotide position in the library, either the first or the second nucleotide position was discarded from the library for failing to add further discriminatory power over the other nucleotide position.

Next, from the reduced binary data set, the polymorphic information content (PIC) of each nucleotide position in the library was calculated based on a modified version of the equation disclosed in Botstein et al., Am. J. Hum. Genet. 32:314-331 (1980). Specifically, for each position in the library, PIC was calculated as [1−Σ(frequency of the assigned first binary value at the position)²+(frequency of assigned second binary value at the position)²)].

All possible pairs of nucleotide positions within the reduced binary data set were then compared, and two calculations were performed: (i) the pairwise sum of binary distribution differences between each nucleotide position in each possible pair; and (ii) the overall mean sum of binary differences in the reduced binary data set based on all of the possible pairs. Where the pairwise sum of (i) was smaller than the overall mean sum of (ii), the nucleotide position with the lower PIC of the two nucleotide positions in the given pair was discarded. Finally, the remaining nucleotide positions were sorted in decreasing order of PIC values, and 10 nucleotide positions within the 5 selected Klebsiella loci were selected for further testing.

### Example 2

### Determination of Klebsiella Sequence Types

The ability to quickly and efficiently type bacteria by clonotyping is particularly valuable in clinical settings (e.g., identifying antibiotic-resistant bacteria in a hospital setting). A useful clonotype should be at least as accurate for predicting relatedness as a standard sequence-based typing scheme, which takes much longer to perform. Prior to testing the clonotype generated in Example 1 on a discovery set of Klebsiella isolates, a reference was established by typing the isolates according to a standard MLST scheme and testing antibiotic resistance.

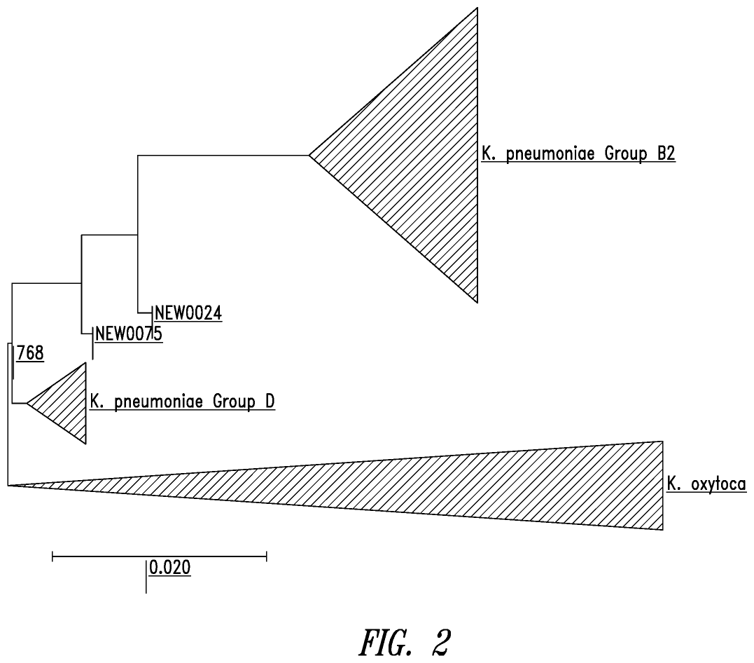

First, a discovery set of 387 clinical extraintestinal Klebsiella spp. isolates was obtained. The five MLST loci described in Example 1 were sequenced for all 387 isolates. A phylogenetic tree was constructed using concatenated sequences of alleles for five MLST loci, gapA (450 bp), infB (318 bp), mdh (477 bp), phoE (420 bp), rpoB (501 bp) (MEGA 7.0 software). In particular, STs that were not found in the klebsiella MLST database (bigsdb.pasteur.fr/klebsiella/klebsiella.html; searched in January 2017) and that differed from a known ST by only one single nucleotide polymorphism (out of 2,166 nucleotides from the combined five MLST loci) were assigned the same number as the known ST. STs that were not found in the MLST database and that differed from a known ST by more than one SNP were termed “new” STs and were each assigned a sequential number using an in-house scheme. STs that belonged to one distinct branch of the phylogenetic tree were also assigned to respective phylogroups (FIG. 2). Since there was no correlation between this assignment method and other published phylogenetic group assignments, distinct K. pneumoniae groups were named by analogy to a known extraintestinal Escherichia coli naming scheme as groups B2 and D. K. oxytoca was named separately.

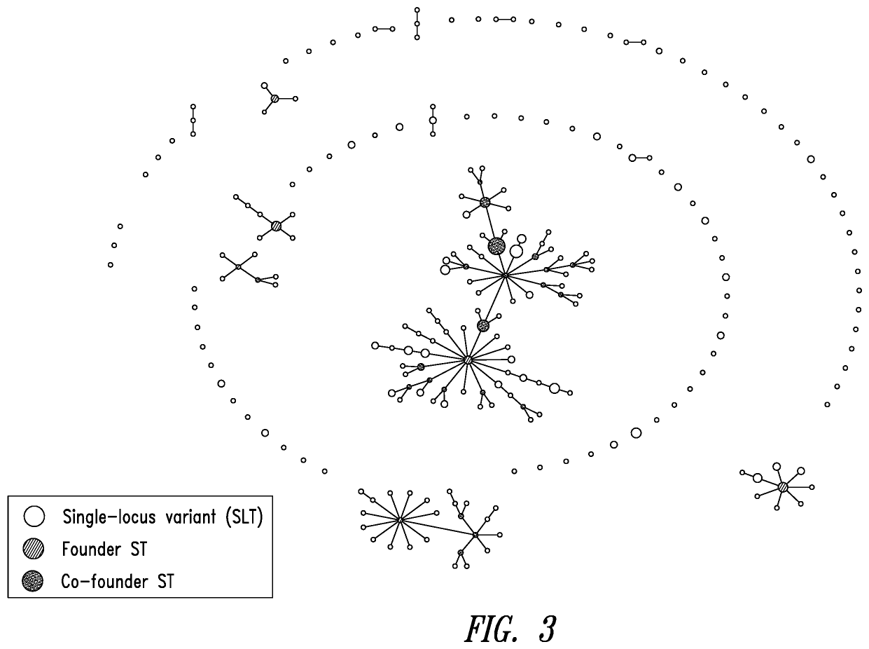

To evaluate the clonal relationship between different STs, a spanning tree of the 387-isolate discovery set was built using eBURST v 3.0 software (eburst.mlst.net/). All neighboring STs that differed in one out of five alleles were assigned to the same clonal complex (CC), named after the founder ST as predicted using eBURST. In CCs with multi-step branching (i.e., where a first ST differs from a second ST at more than one allele and a third, intermediate ST differs from either the first or second ST by one allele), sub-complexes (SC) were assigned wherever there was a founding ST with at least two terminally branched single locus variant STs. The resultant spanning tree is shown in FIG. 3.

### Example 3

### Antibiotic Resistance Profiles of Klebsiella

Next, antibiotic resistance was determined for all 387 Klebsiella isolates in the discovery set. Antibiotic testing was carried out according to the standard disk diffusion method as described in the Clinical and Laboratory Standards Institute (M100 Performance Standards for Antimicrobial Susceptibility Testing, 27th Edition, 2017). The antibiotics tested were: ampicillin (AMP), amoxicillin/clavulanate (A/C), cefazolin (CZ, as representative for first generation cephalosporins), ceftriaxone (CTR, as representative for third generation cephalosporins), trimethoprim/sulfamethoxazole (T/S), ciprofloxacin (CIP, as representative of fluorquinolones), nitrofurantoin (NIT), tetracycline (TET), imipenem (IMI) and ceftazidime vs ceftazidime/clavulanate to determine production of extended-spectrum beta-lactamases (ESBLs). Since almost all Klebsiella isolates are known to be resistant to AMP and sensitive to IMI, TET is not used for treatment of urinary tract infections, and ESBL production is partially reflected in resistance to CTR, these antibiotics were not included for further analysis.

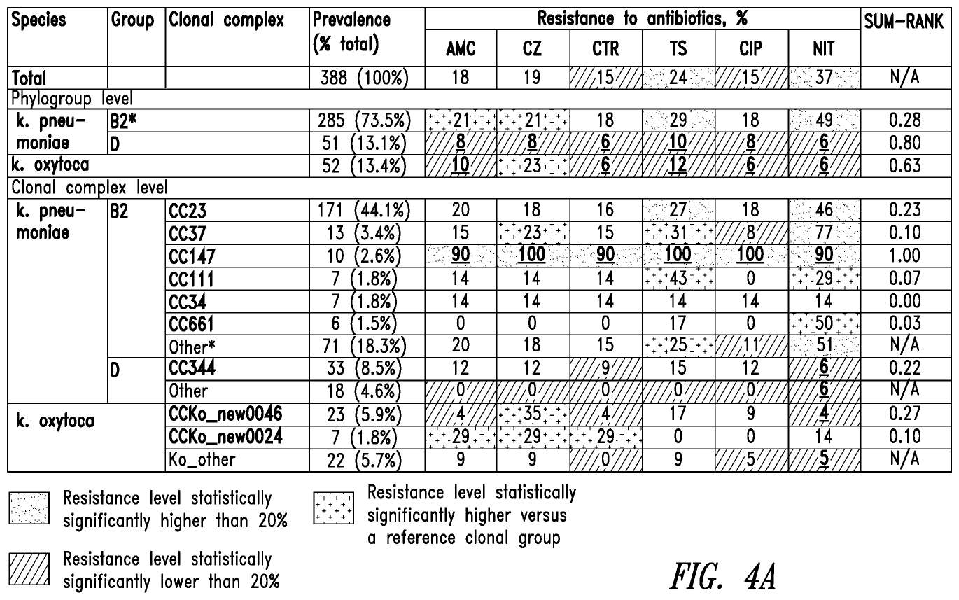

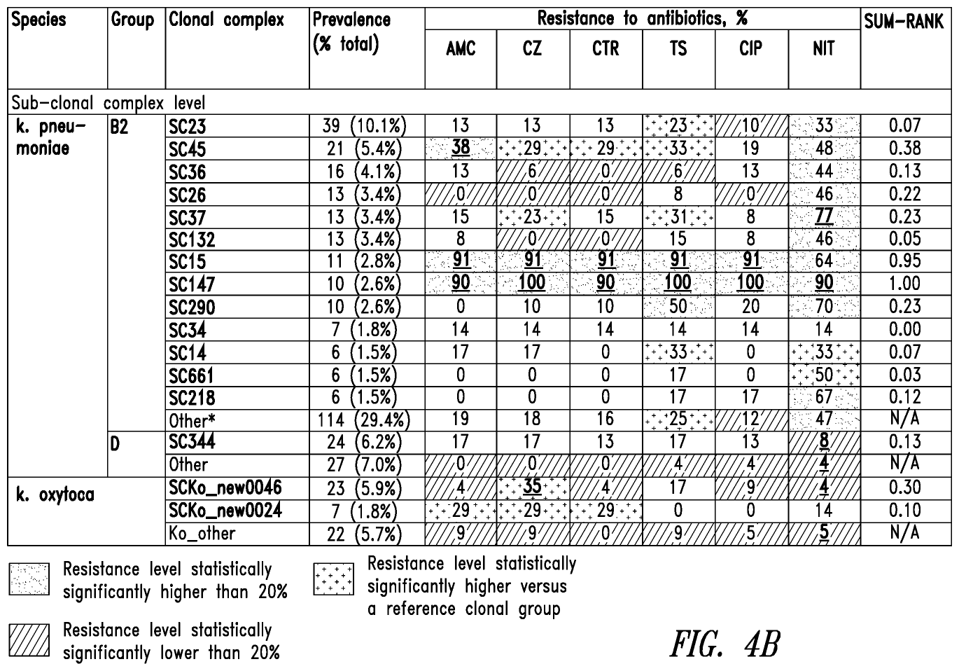

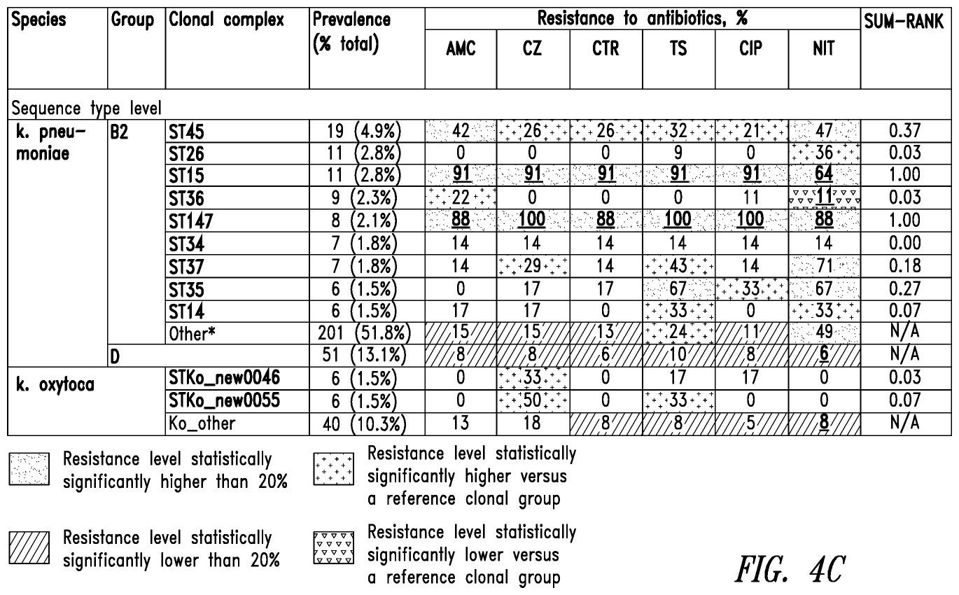

For further analysis, each clonal group—among phylogroups, clonal complexes, clonal sub-complexes and individual STs—was evaluated for the prevalence of resistant isolates to AMC, CZ, CTR, T/S, CIP and NIT antibiotics (FIGS. 4A-4C). Next, clonal groups within each typing scheme were assigned significance weights using four sequential procedures as follows.

First, any clonal group that exhibited resistance rates significantly below or above 20% (as estimated by two-sided one-sample t-test, alpha=0.1) was assigned a +0.5 or −0.5 weight, respectively. The 20% resistance threshold was chosen based on conventional guidelines for antimicrobial treatment adopted by Infectious Diseases Society of America (IDSA, idsociety.org/Guidelines/Patient_Care/IDSA_Practice_Guidelines/Infections_by_Organ_System/Genitourinary/Uncomplicated_Cystitis_and_Pyelonephriti (UTI)/). For the analysis of clone-specific patterns of resistance, intermediate resistance was counted as non-susceptibility.

Second, any clonal group with resistance rates that differed significantly from a reference group within the same clonal grouping scheme (as estimated by multiple logistic regression) was assigned a +0.3 or −0.3 weight for higher and lower resistance, respectively. The reference group was either chosen arbitrarily based on the magnitude of resistance observed in the group or selected as the largest clonal group whose resistance profile was closest to that of overall resistance profile of the discovery set.

Third, any clonal group that exhibited resistance rates above the 20% threshold was assigned a weight of 0.2.

Fourth, each clonal group was ranked for resistance to each antibiotic based on the absolute value of sum of weights, with resistant clonal groups having higher sums due to their relative clinical importance. In this method, only highly resistant clonal groups can be ranked the maximum value of 1.0 per each antibiotic. For each clonal complex, sub-complex, or ST, a clonal group-specific sum-rank (SR) was calculated as an average weight indicating resistance against all six antibiotics of interest (see FIGS. 4A-4C, right-hand-most column).

### Example 4

### Refining Power of Generated Clonotypes

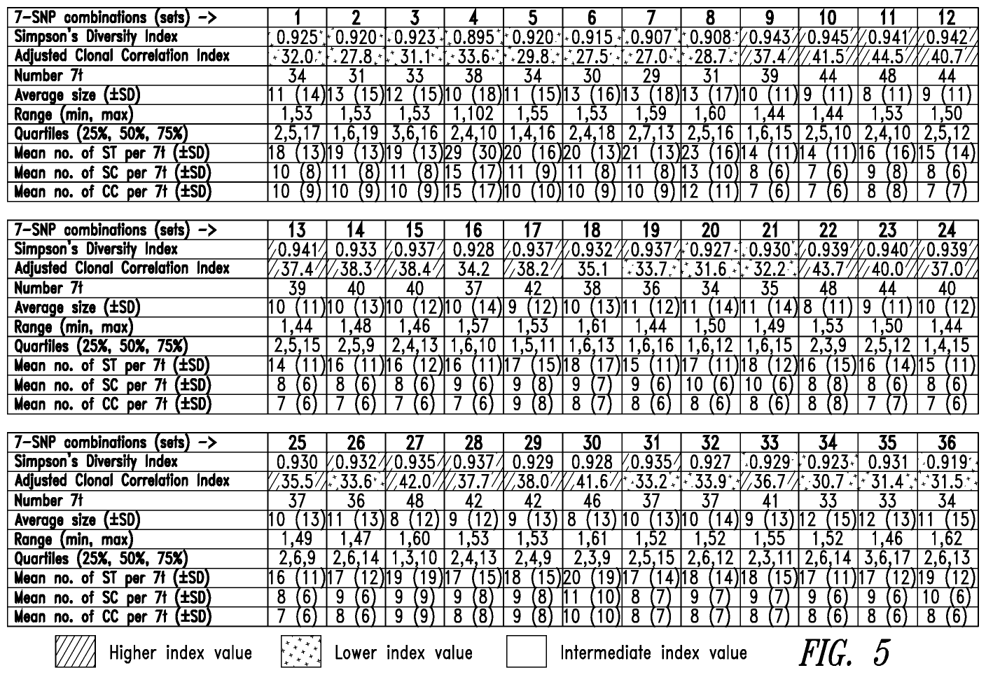

Having established a sequence-typing-based index of relatedness and antibiotic resistance profiles of the discovery set isolates, the ability of the generated clonotype features to predict diversity and resistance was next examined. The 10 SNPs identified using the method described in Example 1 were combined into 36 sets of 7 SNPs each (7 test wells for an 8-well PCR strip tube, plus a well for a control reaction), or “septatypes” or “7ts.” For each of the 36 Its, the following statistics were calculated (see FIG. 5 for results):
Diversity index, based on Simpson's diversity index formula:

DI=1−D=1−Σ(n7t/N)²,
where n is the number of isolates in the 7t and N is the total number of isolates in the set. A larger diversity index indicates higher diversity associated with that clonotype; i.e., an increased probability that two randomly selected Klebsiella isolates will belong to different clonal groups as defined by the clonotype;
Adjusted Clonal Correlation index (ACCI), an in-house measurement of how well the Its correlated with the MLST-determined clonal groups, with emphasis on the clonal groups with pronounced resistance profiles, based on the formula:

ACCI=Σ(P[ST|7t]*SRST)+Σ(P[SC|7t]*SRSC)+Σ(P[CC|7t]*SRCC),
wherein:
   P[ST|7t], P[SC|7t], and P[CC|7t] respectively represent the probability that an isolate from an individual 7t will belong to particular clonal group (i.e., sequence type, subcomplex, or clonal complex), and
   SRST, SRSC, and SRCC are sum-ranks calculated for individual clonal groups (reflecting resistance to antibiotics, see Example 3)
Number of individual Its for each set, their average size, range and quartiles; and
Mean number of individual STs, SCs and CCs per 7t, range and quartile.

Out of 36 possible 7-SNP combinations (or “sets”), 5 were chosen based on having the highest DI and ACCI indexes and further examined manually to confirm that higher resistance or susceptibility of bacteria having the individual 7-SNP combinations to antibiotics is indeed due to the prevalence of specific clonal groups among these types, and not due to random chance.

To select a final 7-SNP set for use in a PCR-based typing assay, the following criteria were used to compare potential primer-binding regions for the SNPs. Several hundred alleles of the sequenced loci (gapA, infB, mdh, phoE and rpoB) were downloaded from multiple available sequence databases. Each SNP's surrounding sequence was analyzed for polymorphisms in the potential primer-binding area(s). SNPs with lowest number of polymorphic sites that could potentially interfere with correct interpretation of PCR reaction were given preference. Additionally, the melting temperature of potential primer binding regions was analyzed to give preference to SNPs that could have primers designed with in-range melting temperature for more robust reactions to allow detection of all 7 SNPs simultaneously.

The 7-SNP combination chosen for further analysis was set no. 12, which included (1) phoE-54, (2) rpoB-130, (3) infB-279, (4) mdh-315, (5) phoE-336, (6) phoE-354 and (7) mdh-429.

### Example 5

### Validation of 7T Test Performance

First, several sets of primers were tested on a training subset of 51 isolates which included 5 E. coli isolates, 1 C. freundii isolate, 1 S. aureus isolates, 2 K. oxytoca isolates, and 42 K. pneumoniae isolates, belonging to different clonal complexes. The final set of primers and reaction conditions were chosen based on their performance on the training set. Additionally, the universal primers for mdh loci were designed to be used as positive control for both K. pneumoniae and K. oxytoca. The names, sequences, and directionality of the final primers are provided below in Table 6. Bold, italicized, underlined bases represent recognized SNPs. Bold, underlined bases represent introduced polymorphisms to make the primer less prone to false positives. Underlined bases (regular font) represent alternative (e.g., A or T or C or G, or a combination thereof) base positions according to the IUPAC nucleotide code (bioinformatics.org/sms/iupac.html). Mdh reference primers SEQ ID NOS:49 and 55 were also generated in-house. (“R/C”=Reverse Complement)

TABLE 6
Final Selected Primers for Clonotyping Klebsiella

| | | Primers | | | | |
|---|---|---|---|---|---|---|
| SNP | | | F primer | F primer | R primer | R primer |
| Pos. | Locus | Dir. | alias | sequence | alias | Sequence |
| 54 | phoE | F | 54-F6A.1 | ACTTCGCCGTCAGCG | 54-R5 | CCCAGGCTTCCGCTT |
| | | | | C [symbol] | | T |
| | | | | (SEQ ID NO: 4) | | (SEQ ID NO: 8) |
| 130 | rpoB | F | 130-F1G | GCCGTATCGTAAAGT | 130-R | TCATCCAGGTTGGAG |
| | | | | GATC [symbol] | | TTCGC |
| | | | | (SEQ ID NO: 20) | | (SEQ ID NO: 25) |
| 279 | infB | F | 279-F1T | CGGCGAGAGCCAGTT | 279-R3 | GCGAACCAGAACGGT |
| | | | | [symbol] | | AG |
| | | | | (SEQ ID NO: 29) | | (SEQ ID NO: 33) |
| 315 | mdh | R | 315-Rev- | TACGATAAAAACAAR | 315-Rev- | CCAGAGTGACCRCCA |
| | | | F | CTGTTCGG | R1 | AT [symbol] |
| | | | | (SEQ ID NO: 41) | | (SEQ ID NO: 42) |
| 336 | phoE | F | 336-F2 | CGAAGGGGTGGGDAG | 336-R | CCTTCGTGGATTACA |
| | | | | TTA [symbol] | | AAATCAACC (R/C) |
| | | | | (SEQ ID NO: 10) | | (SEQ ID NO: 11) |
| 354 | phoE | F | 354-F5C | GCGARGATCTGGTTA | 336-R | CCTTCGTGGATTACA |
| | | | | ACTAGAT [symbol] | | AAATCAACC (R/C) |
| | | | | (SEQ ID NO: 16) | | (SEQ ID NO: 11) |
| 429 | mdh | R | 429-Rev- | CCWTTAYTGTCGCAG | 429-Rev- | TTCGCTTCCACGACA |
| | | | F | ATCCC | R5 | TC [symbol] |
| | | | | (SEQ ID NO: 45) | | (SEQ ID NO: 48) |
| mdh | mdh | N/A | mdh-F2 | GGCGTRGCRAGTAAG | Mdh-R3 | CGTTGTRCTGAAAAA |
| | | | | CCC | | AGCCG |
| | | | | (SEQ ID NO: 49) | | (SEQ ID NO: 55) |

The names, sequences, and directionality of alternate primers are provided below in Table 7.

TABLE 7
Alternate Primers for Clonotyping Klebsiella

| | | Primers | | | | |
|---|---|---|---|---|---|---|
| | | | | | | Alternate |
| SNP | | | F | F primer | R | R primer |
| Pos. | Locus | Dir. | primer | sequence | primer | Sequence |
| 54 | phoE | F | N/A | N/A | 336-R | CCTTCGTGGATT |
| | | | | | | ACAAAATCAACC |
| | | | | | | (R/C) |
| | | | | | | (SEQ ID NO: 11) |
| 130 | rpoB | F | 130- | GCCGTATCGT | N/A | N/A |
| | | | F1A | AAAGTGATC [symbol] | | |
| | | | | (SEQ ID NO: 19) | | |
| 279 | inB | F | N/A | N/A | 279-R5 | ACGACCTTTATC |
| | | | | | | AAGGAAGGA |
| | | | | | | (SEQ ID NO: 35) |
| 315 | mdh | R | 429- | CCWTTAYTGT | N/A | N/A |
| | | | Rev-F | CGCAGATCCC | | |
| | | | | (SEQ ID NO: 45) | | |
| 336 | phoE | F | N/A | N/A | N/A | N/A |
| 354 | phoE | F | N/A | N/A | N/A | N/A |
| 429 | mdh | R | 315- | TACGATAAAA | N/A | N/A |
| | | | Rev-F | ACAARCTGTT | | |
| | | | | CGG | | |
| | | | | (SEQ ID NO: 41) | | |
| mdh | mdh | N/A | N/A | N/A | N/A | N/A |

qPCR reaction volumes were 10 uL, and 10 uM primer stocks were used. SensiMix SYBR No-Rox Kit (Bioline) was used. The reaction mixes for amplifying the indicated SNPs using the primers in Table 1 or Table 2 are provided below in Table 8.

TABLE 8
qPCR Reaction Mixes for Klebsiella Clonotyping

| | | Reaction | | | |
|---|---|---|---|---|---|
| SNP | | F + R | DNA | BioLine Sensimix | H20 |
| Pos. | Locus | (uL) | (uL) | (MM) | (uL) |
| 54 | phoE | 1 + 1 | 1 | 5 | 2 |
| 130 | rpoB | 1 + 1 | 1 | 5 | 2 |
| 279 | infB | 1 + 1 | 1 | 5 | 2 |
| 315 | mdh | 0.5 + 0.5 | 1 | 5 | 3 |
| 336 | phoE | 1 + 1 | 1 | 5 | 2 |
| 354 | phoE | 1.5 + 1 | 1 | 5 | 1.5 |
| 429 | mdh | 0.5 + 0.5 | 1 | 5 | 3 |
| mdh | mdh | 0.5 + 0.5 | 1 | 5 | 3 |

QPCR reactions were performed using a Rotogene Q instrument (72 tubes). Reaction conditions were as shown in Table 9.

TABLE 9
qPCR Reaction Settings for Klebsiella Clonotyping

| | | | Acquisition |
|---|---|---|---|
| Step | Temp (° C.) | Time (s) | channel |
| Initial hot-start | 95 | 600 | None |
| Cycle start (30x repeat) | | | |
| Denaturation | 94 | 5 | None |
| Annealing | 57 | 10 | None |
| Elongation | 72 | 10 | Green |
| Cycle Ends | | | |
| Melting | 75-90 | 0.5 s/° C. | Green |

Threshold reaction settings for amplifying the indicated SNPs are provided below in Table 10.

TABLE 10
Threshold qPCR Reaction Settings for Klebsiella Clonotyping

| SNP | | Reaction | | |
|---|---|---|---|---|
| Pos. | Locus | Delta SNP presence | Delta SNP absence | Tm (° C.) |
| 54 | phoE | 0.7 ± 0.3 (max 1.03) | 5.7 ± 1.07 (min 4.16) | 87.0 ± 0.1 |
| 130 | rpoB | 1.4 ± 1.5 (max 3.02) | 11.2 ± 2.6 (min 6.04) | 81.3 ± 0.3 |
| 279 | infB | 1.58 ± 0.6 (max 2.0) | 12.5 ± 3.6 (min 8.4) | 87.8 ± 0.1 |
| 315 | mdh | 0.8 ± 0.4 (max 1.1) | 12.2 ± 2.9 (min 8.4) | 83.9 ± 0.16 |
| 336 | phoE | 0.1 ± 0.2 (max 0.5) | 15.5 ± 3.4 (min 12.6) | 80.0 ± 0.2 |
| 354 | phoE | 1.4 ± 0.3 (max 1.9) | 13.6 ± 3.4 (min 9.7) | 78.5 ± 0.5 |
| 429 | mdh | 0.8 ± 0.4 (max 1.5) | 11.7 ± 1.9 (min 9.6) | 83.2 ± 0.3 |
| mdh | mdh | NA | NA | 84.2 to 85.6 |

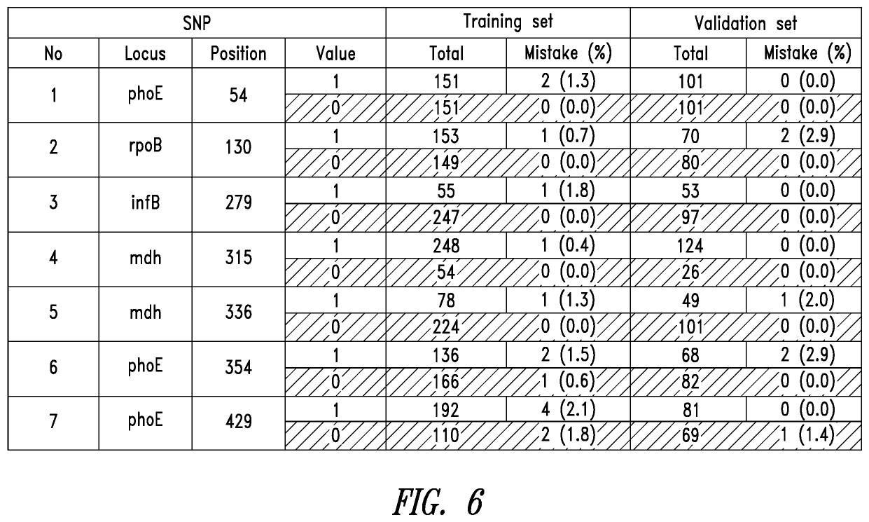

Next, a test set of 302 clinical isolates with SNPs identified via sequencing was used to evaluate the performance of the chosen 7t primers and conditions (FIG. 6). Of this set, 42 isolates belonged to K. oxytoca species and 260 isolates belonged to K. pneumoniae, as predicted by sequencing. Each isolate was tested using the 7t reaction in single replicate, with mistakes between test result and sequence-based prediction ranging from 0 to 2.1% (0.8% on average), and overall 5.3% (16 out of 302 isolates) rate of misidentified clonotypes (FIG. 6).

Finally, a validation set of 150 clinical isolates was first subjected to 7t testing in single replicates, with subsequent sequencing of the loci to determine the correlation between 7t-based and sequence-based results (FIG. 6). mOverall, only 6 isolates (4%) had their clonotypes misidentified during testing.

Thus, the 7t test performed with 95% accuracy for typing isolates in single replicates.

### Example 6

### Validation of Antibiotic Resistance Prediction Based on 7-SNP Test

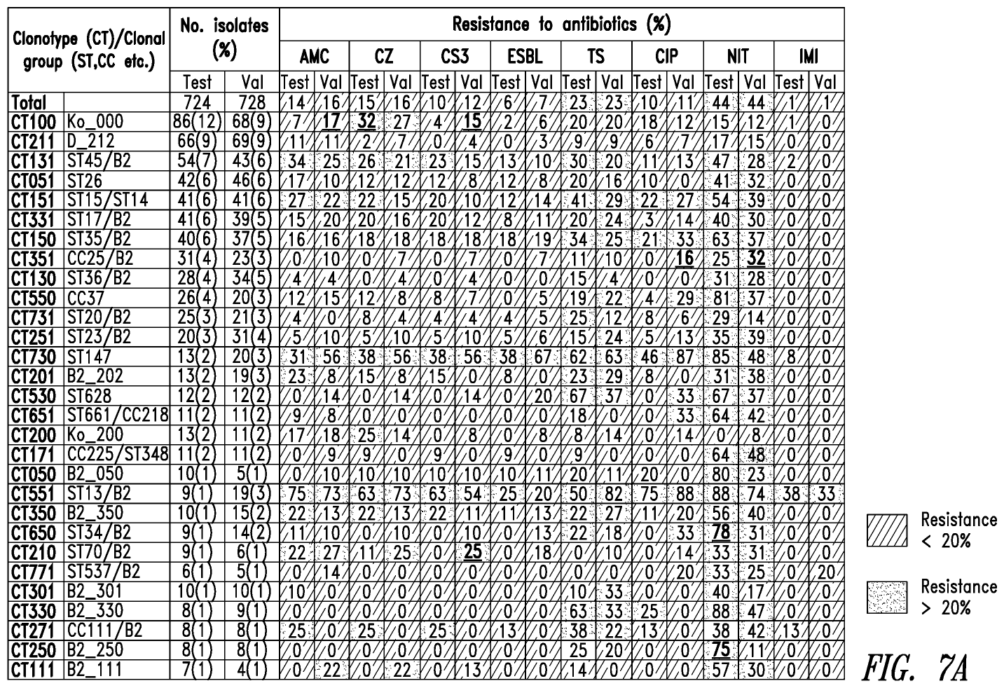

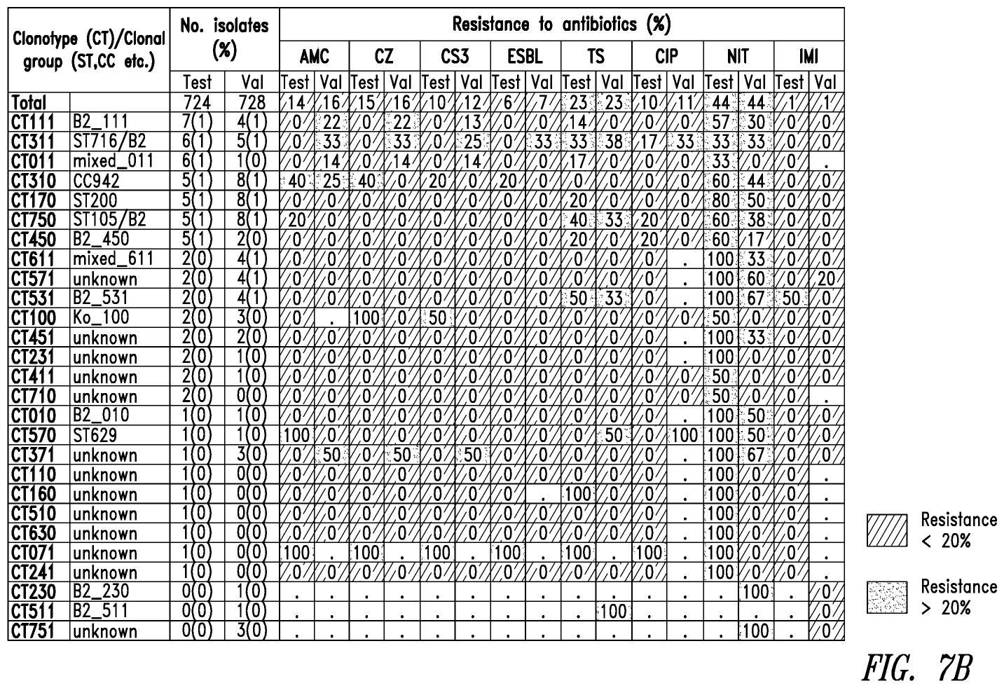

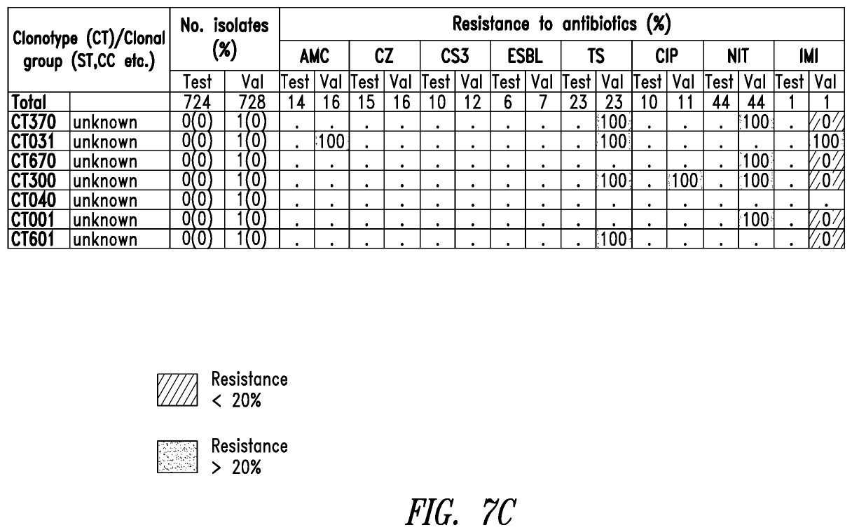

Next, the clonotypes and antibiotic resistance profiles of a set of 1,452 Klebsiella clinical isolates were determined. The set was divided into two subsets based on consecutive isolation dates—training (n=724) and validation (n=728). There was no statistically significant difference in clonotype distribution between the sets. The prevalence of resistant isolates was similar in both sets across all antibiotics tested, and the prevalence of clonotype-specific resistance did not differ significantly across both sets for the majority of clonotypes (FIGS. 7A-7C).

Thus, 7t-based clonotyping successfully predicts the prevalence of resistant isolates among Klebsiella isolates.

### Example 7

### Generation of a Lookup Table

Once identified, an infection characterized by the presence of one or more identified clonotype can be treated with appropriate antibiotics or combinations of antibiotics. To aid in clinical decision-making and to translate clonotype information to treatment selection, Lookup Table 1 was generated. Lookup Table 1 (see Table 1 herein) recommends use or avoidance of antibiotics for treating Klebsiella infections based on the resistance profiles of the collected and tested Its described in the preceding Examples. The information contained in Lookup Table 1 was obtained from urine specimens from patients with Klebsiella infections from several clinics within different regions of the United States. Indications of allowance or non-recommendation were determined based on a 20% resistance threshold to the indicated antibiotic; i.e., the antibiotic is recommended when more than 80% of the tested isolates within the indicated clonotype were susceptible to the indicated antibiotic, and was cautioned against when less than 80% of the isolates were susceptible to the antibiotic.

TABLE 11
Table of Sequences

| | | | | Location | |
|---|---|---|---|---|---|
| SEQ. | | | Direction | of SNP | |
| ID | | | of | (vs. | |
| NO. | Primer name | SNP | Primer | primer) | Primer sequence |
| 1 | phoE54Forw_F1 | 54 | F | F | ACTTCGCCGTCAGCACA |
| 2 | phoE54Forw_F2 | 54 | F | F | ACTTCGCCGTCAGCACG |
| 3 | phoE54Forw_F5 | 54 | F | F | TTCGCCGTCAGCACG |
| 4 | 54-Forw-F6A.1 | 54 | F | F | ACTTCGCCGTCAGCGCA |
| 5 | 54-Forw-F6A.2 | 54 | F | F | TTCGCCTTCAGCGCA |
| 6 | 54-Forw-F6A.3 | 54 | F | F | TTCGCCGTCAGAGCA |
| 7 | phoE54Forw_R | 54 | R | F | CCCAGGCTTCCGCTTTC |
| 8 | 54-Forw-R5 | 54 | R | F | CCCAGGCTTCCGCTTT |
| 9 | phoE336Forw_F1 | 336 | F | F | AAGGGGTGGGDAGTGAG |
| 10 | phoE336Forw-F2 | 336 | F | F | CGAAGGGGTGGGDAGTTAG |
| 11 | phoE336Forw-R | 336 | R | F | GGTTGATTTTGTAATCCACGAAGG |
| 12 | phoE336Rev_F | 336 | F | R | TATCAGTACGACTTCGGTCT |
| 13 | phoE336Rev_R1 | 336 | R | R | GTGRATGTAGTTTACCAGAGCC |
| 14 | phoE354Forw_F1 | 354 | F | F | GTGARGGTCTGGTAAACTACTTC |
| 15 | phoE354Forw_F2 | 354 | F | F | GTGARGGTCTGGTAAACTACATC |
| 16 | 354-Forw-F5C | 354 | F | F | GCGARGATCTGGTTAACTAGATC |
| 17 | phoE354Forw_F5T | 354 | F | F | GCGARGATCTGGTTAACTACGTT |
| 18 | 354-Forw-F5T | 354 | F | F | GCGARGATCTGGTTAACTAGATT |
| 19 | 130-F1A | 130 | F | F | GCCGTATCGTAAAGTGATCA |
| 20 | rpoB130Forw-F1 | 130 | F | F | GCCGTATCGTAAAGTGATCG |
| 21 | rpoB130Forw_F2 | 130 | F | F | GCCGTATCGTAAAGTGACCG |
| 22 | rpoB130Forw_F3 | 130 | F | F | GCCGTATCGTAAAGTGAACA |
| 23 | 130-F5A | 130 | F | F | GCCGTATCGTAAAGTGATGA |
| 24 | 130-F5G | 130 | F | F | GCCGTATCGTAAAGTGATGG |
| 25 | rpoB130Forw-R | 130 | R | F | TCATCCAGGTTGGAGTTCGC |
| 26 | 130-R5 | 130 | R | F | GCGAAACTCCAACCTGGAT |
| 27 | 130-R6 | 130 | R | F | CTGCCGTAGCAAAGGCGAAT |
| 28 | infB279Forw_F1 | 279 | F | F | CGGCGAGAGCCAGTTC |
| 29 | 279-Forw_F1T | 279 | F | F | CGGCGAGAGCCAGTTT |
| 30 | infB279Forw_F2 | 279 | F | F | CGGCGAGAGCCAGATC |
| 31 | 279-Forw_F2T | 279 | F | F | CGGCGAGAGCCAGATT |
| 32 | infB279Forw_R | 279 | R | F | CTGCTGGAGGCTGAAGTTCTT |
| 33 | infB279Forw_r3 | 279 | R | F | GCGAACCAGAACGGTAG |
| 34 | infB279Forw_r4 | 279 | R | F | CGGACCACGACCTTTATC |
| 35 | infB279Forw_r5 | 279 | R | F | ACGACCTTTATCAAGGAAGGA |
| 36 | infB279Rev_F | 279 | F | R | AARATGGATAAGCCAGAAGC |
| 37 | infB279Rev_R1 | 279 | R | R | TTTCGCTGAAACGTGGAYG |
| 38 | infB279Rev_R2 | 279 | R | R | TTTCGCTGAAACGTGGTYG |
| 39 | infB279Rev_R3 | 279 | R | R | TTTCGCTGAAACGTGGACG |
| 40 | infB279Rev_R4 | 279 | R | R | TTTCGCTGAAACGTGGTCG |
| 41 | mdh315Rev-F | 315 | F | R | TACGATAAAAACAARCTGTTCGG |
| 42 | 315-Rev-R1 | 315 | R | R | CCAGAGTGACCRCCAATG |
| 43 | mdh315Rev_R2 | 315 | R | R | CCAGAGTGACCRCCATTG |
| 44 | 315-Rev-R5 | 315 | R | R | CCAGAGTGACCTCCAATG |
| 45 | mdh429Rev-F | 429 | F | R | CCWTTAYTGTCGCAGATCCC |
| 46 | mdh429Rev_R1 | 429 | R | R | CTTTCGCTTCCACGACTTCG |
| 47 | mdh429Rev_R2 | 429 | R | R | CTTTCGCTTCCACGACTACG |
| 48 | 429-Rev-R5 | 429 | R | R | TTCGCTTCCACGACATCG |
| 49 | KlSpMDH-F2 | ref | F | — | GGCGTRGCRAGTAAGCCC |
| 50 | KlSpMDH-F3 | ref | F | — | GGCGTRGCRCGTAAGACC |
| 52 | KlSpMDH-F4 | ref | F | — | ATGAAAGTTGCAGTCCT |
| 53 | KlSpMDH-F5 | ref | F | — | GCGTGGCGGTTGATCT |
| 54 | KlSpMDH-R2 | ref | R | — | GCTTTTTTCAGYACTTCTKGCG |
| 55 | KlSpMDH-R3 | ref | R | — | CGGCTTTTTTCAGYACTTCKGC |
| 56 | KlSpMDH-R4 | ref | R | — | CGGCTTTTTTCAGYACATCKGC |
| 57 | KlSpMDH-R5 | ref | R | — | ATGTCGTACAACGAGAG |
| 58 | KlSpMDH-R6 | ref | R | — | AGATCAACCGCCACGC |
| 59 | KlSpMDH-R7 | ref | R | — | GGAKATCAGCACYACATC |
| 60 | rpoB130Forw_R2 | 130 | R | F | ATCTTCTACGAAGTGGCCGTT |
| 61 | 354-F6C | 354 | F | F | GCGATGATCTGGTTAACTAGATC |

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including U.S. Provisional Patent Application No. 62/668,042, filed May 7, 2018, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A method for generating a clonotype, the method comprising:
(a) generating a full binary data set from a nucleotide position library, wherein the full binary data set comprises, for each nucleotide position in the library,
(1) an assigned first binary value to a nucleotide base that appears most frequently at the position within the library, and
(2) an assigned second binary value to all other nucleotide bases that appear at the position within the library,
wherein the first and second assigned binary values are different; and
wherein the nucleotide position library comprises an aligned, concatenated nucleic acid sequence set obtained from one or more loci in a genome, in which (i) nucleotide positions with a gap and (ii) nucleotide positions that are monomorphic in the sequence set are discarded;
(b) generating a reduced binary data set, comprising discarding from the full binary data set one nucleotide position from each pair of nucleotide positions having identical or reverse-identical binary distribution patterns;
(c) generating a Polymorphic Information Content (PIC) of each nucleotide position in the reduced binary data set, wherein:
PIC=[1−Σ(frequency of the assigned first binary value at the position)²+(frequency of assigned second binary value at the position)²)];
(d) identifying all possible pairs of nucleotide positions in the reduced binary data set;
(e) generating a PIC differential, wherein the PIC differential comprises:
(i) a pairwise sum of binary distribution differences between two nucleotide positions of a pair for each of the possible pairs of the nucleotide positions in the reduced binary data set,
(ii) an overall mean sum of binary distribution differences in the reduced binary data set based on all possible pairs of the nucleotide positions in the reduced binary data set,
wherein the nucleotide position with the lower PIC of the two nucleotide positions in a pair is discarded when the pairwise sum of binary distribution differences of (i) is smaller than the overall mean sum of the binary distribution differences of (ii); and
(f) selecting non-discarded nucleotide positions to generate a clonotype.

2. The method of claim 1, comprising, following (e)(ii) and prior to (f), ordering the non-discarded nucleotide positions according to PIC value.

3. The method of claim 1 or 2, wherein the nucleotide position library comprises nucleic acid sequences from one or more one allele of the one or more locus.

4. The method of any one of claims 1-3, wherein the nucleotide position library comprises nucleic acid sequences from a bacterium; a human cell, optionally a T cell; a tumor; a non-human animal; or a plant.

5. The method of claim 4, wherein the nucleic acid sequences are selected from the group consisting of:
Acinetobacter baumannii; Actinomyces israelii; Actinomyces gerencseriae; Anaplasma species; Ancylostoma braziliense; Angiostrongylus; Anisakis; Arcanobacterium haemolyticum; Junin virus; Ascaris lumbricoides; Aspergillus species; an Astroviridae family member; Anaplasma phagocytophilum; Actinomycetoma sp.; Babesia sp.; Bacillus anthracis; Bacillus cereus; Bacillus sp.; Bacteroides sp.; Balantidium coli; Bartonella; Batrachochytrium dendrabatidis; Baylisascaris species; Blastocystis sp.; Blastomyces dermatitidis; Bartonella bacilliformis; Bartonella henselae; Borrelia burgdorferi, Borrelia hermsii, Borrelia recurrentis, Borrelia garinii, Borrelia afzelii; Bordetella pertussis; Brucella sp.; Brevibacterium sp.; Burkholderia mallei, Burkholderia pseudomallei, Burkholderia cepacia; Campylobacter sp.; Candida sp.; Capillaria philippinensis, Capillaria hepatica, Capillaria aerophila; Chlamydia trachomatis, Chlamydophila pneumoniae, Chlamydophila psittaci; Citrobacter freundii, Citrpbacter koserii, Citrobacter sedlakii and Citrobacter sp.; Clonorchis sinensis; Corynebacterium diphtheria and Corynebacterium sp; Clostridium botulinum; Clostridium difficile; Clostridium tetani; Clostridium perfringens; Clostridium sp; Coxiella burnetii; Cryptococcus neoformans; Cryptosporidium sp.; Cyclospora cayetanensis; Escherichia coli; Escherichia coli O157:H7, Escherichia coli O111; Escherichia coli O104:H4; Ehrlichia ewingii; Ehrlichia chaffeensis; Ehrlichia sp.; Echinococcus sp.; Enterococcus faecalis; Enterococcus faecium; Enterococcus sp.; Entamoeba histolytica; Enterobacter aerogenes; Enterobacter cloacae; Fusobacterium sp.; Fonsecaea pedrosoi; Francisella tularensis; Geotrichum candidum; Haemophilus ducreyi; Haemophilus influenza; Helicobacter pylori; Klebsiella pneumoniae; Klebsiella oxytoca; Klebsiella granulomatis; Klebsiella variicola; Klebsiella sp.; Kingella kingae; Kluyvera ascorbata; Legionella pneumophila; Leptospira sp.; Listeriamonocytogenes; Mycobacterium tuberculosis; Mycobacterium ulcerans; Mycobacterium leprae; Mycobacterium lepromatosis; Mycoplasma pneumoniae; Moraxella sp.; Morganella morganii; Neisseria gonorrhoeae; Neisseria meningitides; Nocardia asteroids; Piedraia hortae; Pantoea agglomerans, Pseudomonas aeruginosa; Pseudomonas sp., Proteus mirabilis; Proteus sp.; Pasteurella sp.; Prevotella sp.; Propionibacterium propionicus; Rickettsia rickettsia; Rickettsia prowazekii; Rickettsia typhi; Rickettsia akari; Raoultella ornithinolytica; Raoultella planticola; Raoultella sp.; Streptococcus pneumoniae; Streptococcus pyogenes; Streptococcus agalactiae; Streptococcus sp.; Salmonella enterica subsp. Enterica; Salmonella serovar typhi; Salmonella sp.; Shigella sp.; Staphylococcus aureus; Staphylococcus saprophyticus; Staphylococcus epidermidis; Staphylococcus haemolyticus; Staphylococcus sp.; Serratia marcensens; Serratia liquefaciens; Serratia grimesii; Serratia maltophilia; Trypanosoma brucei; Trichosporon beigelii; Ureaplasma urealyticum; Vibrio cholera, Vibrio vulnificus, Vibrio parahaemolyticus; Yersinia pestis; Yersinia enterocolitica; and Yersinia pseudotuberculosis.

6. The method of any one of claims 1-5, wherein generating the clonotype comprises selecting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 500, 1000, 10,000, or more nucleotide positions, each having a PIC value above a pre-determined threshold PIC value.

7. The method of claim 6, wherein selecting the one or more nucleotide positions with the PIC values comprises selecting 5, 6, 7, 8, 9, or 10 nucleotide positions, preferably 7 nucleotide positions.

8. The method of any one of claims 1-7, further comprising testing the generated clonotype on a sample comprising nucleic acids from the organism or cell type of interest, wherein the organism or cell type of interest is of one or more predetermined sequence type, wherein the testing comprises
(a) performing an amplification reaction on the sample using forward and reverse primers for the clonotype nucleotide positions; and
(b) comparing results from the amplification reaction with the one or more predetermined sequence types.

9. The method of claim 8, wherein the nucleic acid amplification reaction comprises a polymerase chain reaction (PCR), optionally a quantitative polymerase chain reaction (qPCR).

10. A method for determining the presence or absence of a single nucleotide polymorphism (SNP) in Klebsiella, the method comprising performing a nucleic acid amplification process on DNA isolated from Klebsiella obtained from a patient sample, wherein the nucleic acid amplification process comprises use of forward and reverse primer pairs specific for phoE54, rpoB130, infB279, mdh315, phoE336, phoE354, and mdh429, and determining the presence or absence of one or more of the phoE54, rpoB130, infB279, mdh315, phoE336, phoE354, and mdh429 SNPs.

11. The method of claim 10, wherein the primer pairs comprise one or more of the following primer pairs:
(a) forward primer and reverse primer pairs for at least seven Klebsiella single nucleotide polymorphisms (SNPs), wherein the SNPs comprise phoE54, rpoB130, infB279, mdh315, phoE336, phoE354, and mdh429, and wherein the primer pairs comprise one or more of the following primer pairs:
(i) a phoE54 forward primer comprising or consisting of the nucleic acid sequence of SEQ ID NO:4 and a phoE54 reverse primer comprising or consisting of the nucleic acid sequence of SEQ ID NO:8 or SEQ ID NO:11,
(ii) a rpoB130 forward primer comprising or consisting of the nucleic acid sequence of SEQ ID NO:20 or SEQ ID NO:19 and a rpoB130 reverse primer comprising or consisting of the nucleic acid sequence of SEQ ID NO:25 or SEQ ID NO:60,
(iii) a infB279 forward primer comprising or consisting of the nucleic acid sequence of SEQ ID NO:29 and a infB279 reverse primer comprising or consisting of the nucleic acid sequence of SEQ ID NO:33 or SEQ ID NO:35,
(iv) a mdh315 forward primer comprising or consisting of the nucleic acid sequence of SEQ ID NO:41 or SEQ ID NO:49 and a mdh315 reverse primer comprising or consisting of the nucleic acid sequence of SEQ ID NO:42,
(v) a phoE336 forward primer comprising or consisting of the nucleic acid sequence of SEQ ID NO:10 and a phoE336 reverse primer comprising or consisting of the nucleic acid sequence of SEQ ID NO:11,
(vi) a phoE354 forward primer comprising the nucleic acid sequence of SEQ ID NO:16 and a phoE354 reverse primer comprising the nucleic acid sequence of SEQ ID NO:11, and
(vii) a mdh429 forward primer comprising or consisting of the nucleic acid sequence of any one of SEQ ID NOs:45, 41, or 49, and a mdh429 reverse primer comprising or consisting of the nucleic acid sequence of SEQ ID NO:48.

12. The method of claim 11, wherein the primer pairs comprise:
(i) a phoE54 forward primer comprising or consisting of the nucleic acid sequence of SEQ ID NO:4 and a phoE54 reverse primer comprising or consisting of the nucleic acid sequence of SEQ ID NO:8 or SEQ ID NO:11,
(ii) a rpoB130 forward primer comprising or consisting of the nucleic acid sequence of SEQ ID NO:20 or SEQ ID NO:19 and a rpoB130 reverse primer comprising or consisting of the nucleic acid sequence of SEQ ID NO:25 or SEQ ID NO:60,
(iii) a infB279 forward primer comprising or consisting of the nucleic acid sequence of SEQ ID NO:29 and a infB279 reverse primer comprising or consisting of the nucleic acid sequence of SEQ ID NO:33 or SEQ ID NO:35,
(iv) a mdh315 forward primer comprising or consisting of the nucleic acid sequence of SEQ ID NO:41 or SEQ ID NO:49 and a mdh315 reverse primer comprising or consisting of the nucleic acid sequence of SEQ ID NO:42,
(v) a phoE336 forward primer comprising or consisting of the nucleic acid sequence of SEQ ID NO:10 and a phoE336 reverse primer comprising or consisting of the nucleic acid sequence of SEQ ID NO:11,
(vi) a phoE354 forward primer comprising the nucleic acid sequence of SEQ ID NO:16 and a phoE354 reverse primer comprising the nucleic acid sequence of SEQ ID NO:11, and
(vii) a mdh429 forward primer comprising or consisting of the nucleic acid sequence of any one of SEQ ID NOs:45, 41, or 49, and and a mdh429 reverse primer comprising or consisting of the nucleic acid sequence of SEQ ID NO:48.

13. A method for determining antibiotic susceptibility of Klebsiella, the method comprising:
(a) amplifying polynucleotide fragments from a Klebsiella genome using forward and reverse primer pairs specific for at least seven different Klebsiella single nucleotide polymorphisms (SNPs), wherein the at least seven different SNPs comprise phoE54, rpoB130, infB279, mdh315, phoE336, phoE354, and mdh429, and wherein the primer pairs comprise one or more of the following primer pairs:
(a) forward primer and reverse primer pairs for at least seven Klebsiella single nucleotide polymorphisms (SNPs), wherein the SNPs comprise phoE54, rpoB130, infB279, mdh315, phoE336, phoE354, and mdh429, and wherein the primer pairs comprise one or more of the following primer pairs:
(i) a phoE54 forward primer comprising or consisting of the nucleic acid sequence of SEQ ID NO:4 and a phoE54 reverse primer comprising or consisting of the nucleic acid sequence of SEQ ID NO:8 or SEQ ID NO:11,
(ii) a rpoB130 forward primer comprising or consisting of the nucleic acid sequence of SEQ ID NO:20 or SEQ ID NO:19 and a rpoB130 reverse primer comprising or consisting of the nucleic acid sequence of SEQ ID NO:25 or SEQ ID NO:60,
(iii) a infB279 forward primer comprising or consisting of the nucleic acid sequence of SEQ ID NO:29 and a infB279 reverse primer comprising or consisting of the nucleic acid sequence of SEQ ID NO:33 or SEQ ID NO:35,
(iv) a mdh315 forward primer comprising or consisting of the nucleic acid sequence of SEQ ID NO:41 or SEQ ID NO:49 and a mdh315 reverse primer comprising or consisting of the nucleic acid sequence of SEQ ID NO:42,
(v) a phoE336 forward primer comprising or consisting of the nucleic acid sequence of SEQ ID NO:10 and a phoE336 reverse primer comprising or consisting of the nucleic acid sequence of SEQ ID NO:11,
(vi) a phoE354 forward primer comprising the nucleic acid sequence of SEQ ID NO:16 and a phoE354 reverse primer comprising the nucleic acid sequence of SEQ ID NO:11, and
(vii) a mdh429 forward primer comprising or consisting of the nucleic acid sequence of any one of SEQ ID NOs:45, 41, or 49, and a mdh429 reverse primer comprising or consisting of the nucleic acid sequence of SEQ ID NO:48;
(b) detecting the presence or absence of one or more of the at least seven SNPs in the Klebsiella genome to identify the Klebsiella clonotype; and
(c) comparing the Klebsiella clonotype to a Lookup Table to determine the Klebsiella susceptibility to one or more antibiotics.

14. The method of claim 13, wherein the Lookup Table is Lookup Table 1.

15. The method of claim 13 or 14, wherein the primer pairs comprise:
(i) a phoE54 forward primer comprising or consisting of the nucleic acid sequence of SEQ ID NO:4 and a phoE54 reverse primer comprising or consisting of the nucleic acid sequence of SEQ ID NO:8 or SEQ ID NO:11,
(ii) a rpoB130 forward primer comprising or consisting of the nucleic acid sequence of SEQ ID NO:20 or SEQ ID NO:19 and a rpoB130 reverse primer comprising or consisting of the nucleic acid sequence of SEQ ID NO:25 or SEQ ID NO:60,
(iii) a infB279 forward primer comprising or consisting of the nucleic acid sequence of SEQ ID NO:29 and a infB279 reverse primer comprising or consisting of the nucleic acid sequence of SEQ ID NO:33 or SEQ ID NO:35,
(iv) a mdh315 forward primer comprising or consisting of the nucleic acid sequence of SEQ ID NO:41 or SEQ ID NO:49 and a mdh315 reverse primer comprising or consisting of the nucleic acid sequence of SEQ ID NO:42,
(v) a phoE336 forward primer comprising or consisting of the nucleic acid sequence of SEQ ID NO:10 and a phoE336 reverse primer comprising or consisting of the nucleic acid sequence of SEQ ID NO:11,
(vi) a phoE354 forward primer comprising the nucleic acid sequence of SEQ ID NO:16 and a phoE354 reverse primer comprising the nucleic acid sequence of SEQ ID NO:11, and
(vii) a mdh429 forward primer comprising or consisting of the nucleic acid sequence of any one of SEQ ID NOs:45, 41, or 49, and a mdh429 reverse primer comprising or consisting of the nucleic acid sequence of SEQ ID NO:48.

16. A method for treating a Klebsiella infection in a patient, the method comprising administering to a patient in need thereof an effective amount of one or more antibiotic, wherein the Klebsiella infecting the patient is known to be susceptible to the one or more administered antibiotic as determined by the method of any one of claims 13-15.

17. The method of claim 16, wherein the one or more antibiotics are selected from ampicillin (AMP), amoxicillin/clavulanate (A/C), a first-generation cephalosporin, a third generation cephalosporin, trimethoprim/sulfamethoxazole (T/S), a fluorquinolone, nitrofurantoin (NIT), tetracycline (TET), imipenem (IMI), ceftazidime/clavulanate, or any combination thereof.

18. The method of claim 17, wherein the first-generation cephalosporin comprises cefazolin (CZ).

19. The method of claim 17, wherein the third-generation cephalosporin comprises ceftriaxone (CTR).

20. The method of claim 17, wherein the fluorquinolone comprises ciproflaxin (CIP).

21. The method of any one of claims 10-20 wherein the Klebsiella is from a patient sample, an invasive medical instrument, or a patient-accessible surface in a healthcare or elder care setting.

22. The method of claim 21, wherein the Klebsiella is from a patient sample selected from the group consisting of urine, a fecal swab, a wound swab, blood, saliva, sputum, a nasal swab, a tracheal swab, an abscess aspirate, and a skin swipe.

23. The method of claim 21, wherein the patient sample comprises sputum.

24. The method of any one of claims 21-23, wherein the patient sample was fractionated to separate the bacterial components from non-bacterial nucleic acids, ureas, and solids.

25. The method of claim 24, wherein the fractionated bacteria were lysed prior to performing the amplifying step.

26. A kit, comprising:
(i) a phoE54 forward primer comprising or consisting of the nucleic acid sequence of SEQ ID NO:4 and a phoE54 reverse primer comprising or consisting of the nucleic acid sequence of SEQ ID NO:8 or SEQ ID NO:11,
(ii) a rpoB130 forward primer comprising or consisting of the nucleic acid sequence of SEQ ID NO:20 or SEQ ID NO:19 and a rpoB130 reverse primer comprising or consisting of the nucleic acid sequence of SEQ ID NO:25 or SEQ ID NO:60,
(iii) a infB279 forward primer comprising or consisting of the nucleic acid sequence of SEQ ID NO:29 and a infB279 reverse primer comprising or consisting of the nucleic acid sequence of SEQ ID NO:33 or SEQ ID NO:35,
(iv) a mdh315 forward primer comprising or consisting of the nucleic acid sequence of SEQ ID NO:41 or SEQ ID NO:49 and a mdh315 reverse primer comprising or consisting of the nucleic acid sequence of SEQ ID NO:42,
(v) a phoE336 forward primer comprising or consisting of the nucleic acid sequence of SEQ ID NO:10 and a phoE336 reverse primer comprising or consisting of the nucleic acid sequence of SEQ ID NO:11,
(vi) a phoE354 forward primer comprising the nucleic acid sequence of SEQ ID NO:16 and a phoE354 reverse primer comprising the nucleic acid sequence of SEQ ID NO:11, and
(vii) a mdh429 forward primer comprising or consisting of the nucleic acid sequence of any one of SEQ ID NOs:45, 41, or 49, and a mdh429 reverse primer comprising or consisting of the nucleic acid sequence of SEQ ID NO:48;
(b) optional additional reagents for performing an nucleic acid amplification reaction;
(c) an optional Lookup Table; and
(d) an optional instruction for identifying a Klebsiella clonotype and determining the Klebsiella susceptibility to one or more antibiotics.

27. The kit of claim 26, wherein the Lookup Table is Lookup Table 1.

28. The kit of claim 26 or 27, wherein the primer pairs comprise:
(i) a phoE54 forward primer comprising or consisting of the nucleic acid sequence of SEQ ID NO:4 and a phoE54 reverse primer comprising or consisting of the nucleic acid sequence of SEQ ID NO:8 or SEQ ID NO:11,
(ii) a rpoB130 forward primer comprising or consisting of the nucleic acid sequence of SEQ ID NO:20 or SEQ ID NO:19 and a rpoB130 reverse primer comprising or consisting of the nucleic acid sequence of SEQ ID NO:25 or SEQ ID NO:60,
(iii) a infB279 forward primer comprising or consisting of the nucleic acid sequence of SEQ ID NO:29 and a infB279 reverse primer comprising or consisting of the nucleic acid sequence of SEQ ID NO:33 or SEQ ID NO:35,
(iv) a mdh315 forward primer comprising or consisting of the nucleic acid sequence of SEQ ID NO:41 or SEQ ID NO:49 and a mdh315 reverse primer comprising or consisting of the nucleic acid sequence of SEQ ID NO:42,
(v) a phoE336 forward primer comprising or consisting of the nucleic acid sequence of SEQ ID NO:10 and a phoE336 reverse primer comprising or consisting of the nucleic acid sequence of SEQ ID NO:11,
(vi) a phoE354 forward primer comprising the nucleic acid sequence of SEQ ID NO:16 and a phoE354 reverse primer comprising the nucleic acid sequence of SEQ ID NO:11, and
(vii) a mdh429 forward primer comprising or consisting of the nucleic acid sequence of any one of SEQ ID NOs:45, 41, or 49 and a mdh429 reverse primer comprising or consisting of the nucleic acid sequence of SEQ ID NO:48.

29. The kit of any one of claims 26-28, wherein at least two of the primer pairs selected from (a)(i)-(a)(vii) are mixed in a single container.

30. The method of any one of claims 10-25 or the kit of any one of claims 26-29, further comprising a mdh forward primer comprising or consisting of the nucleotide sequence of SEQ ID NO:49 and a mdh reverse primer comprising or consisting of the nucleotide sequence of SEQ ID NO:55.


SEQUENCE LISTING (61 sequences)

SEQ ID NO: 1 - DNA, 17 bp, Artificial Sequence
  Primer sequence
acttcgccgtcagcaca

SEQ ID NO: 2 - DNA, 17 bp, Artificial Sequence
  Primer sequence
acttcgccgtcagcacg

SEQ ID NO: 3 - DNA, 15 bp, Artificial Sequence
  Primer sequence
ttcgccgtcagcacg

SEQ ID NO: 4 - DNA, 17 bp, Artificial Sequence
  Primer sequence
acttcgccgtcagcgca

SEQ ID NO: 5 - DNA, 15 bp, Artificial Sequence
  Primer sequence
ttcgccttcagcgca

SEQ ID NO: 6 - DNA, 15 bp, Artificial Sequence
  Primer sequence
ttcgccgtcagagca

SEQ ID NO: 7 - DNA, 17 bp, Artificial Sequence
  Primer sequence
cccaggcttccgctttc

SEQ ID NO: 8 - DNA, 16 bp, Artificial Sequence
  Primer sequence
cccaggcttccgcttt

SEQ ID NO: 9 - DNA, 17 bp, Artificial Sequence
  Primer sequence
aaggggtgggdagtgag

SEQ ID NO: 10 - DNA, 19 bp, Artificial Sequence
  Primer sequence
cgaaggggtgggdagttag

SEQ ID NO: 11 - DNA, 24 bp, Artificial Sequence
  Primer sequence
ggttgattttgtaatccacgaagg

SEQ ID NO: 12 - DNA, 20 bp, Artificial Sequence
  Primer sequence
tatcagtacgacttcggtct

SEQ ID NO: 13 - DNA, 22 bp, Artificial Sequence
  Primer sequence
gtgratgtagtttaccagagcc

SEQ ID NO: 14 - DNA, 23 bp, Artificial Sequence
  Primer sequence
gtgarggtctggtaaactacttc

SEQ ID NO: 15 - DNA, 23 bp, Artificial Sequence
  Primer sequence
gtgarggtctggtaaactacatc

SEQ ID NO: 16 - DNA, 23 bp, Artificial Sequence
  Primer sequence
gcgargatctggttaactagatc

SEQ ID NO: 17 - DNA, 23 bp, Artificial Sequence
  Primer sequence
gcgargatctggttaactacgtt

SEQ ID NO: 18 - DNA, 23 bp, Artificial Sequence
  Primer sequence
gcgargatctggttaactagatt

SEQ ID NO: 19 - DNA, 20 bp, Artificial Sequence
  Primer sequence
gccgtatcgtaaagtgatca

SEQ ID NO: 20 - DNA, 20 bp, Artificial Sequence
  Primer sequence
gccgtatcgtaaagtgatcg

SEQ ID NO: 21 - DNA, 20 bp, Artificial Sequence
  Primer sequence
gccgtatcgtaaagtgaccg

SEQ ID NO: 22 - DNA, 20 bp, Artificial Sequence
  Primer sequence
gccgtatcgtaaagtgaaca

SEQ ID NO: 23 - DNA, 20 bp, Artificial Sequence
  Primer sequence
gccgtatcgtaaagtgatga

SEQ ID NO: 24 - DNA, 20 bp, Artificial Sequence
  Primer sequence
gccgtatcgtaaagtgatgg

SEQ ID NO: 25 - DNA, 20 bp, Artificial Sequence
  Primer sequence
tcatccaggttggagttcgc

SEQ ID NO: 26 - DNA, 19 bp, Artificial Sequence
  Primer sequence
gcgaaactccaacctggat

SEQ ID NO: 27 - DNA, 20 bp, Artificial Sequence
  Primer sequence
ctgccgtagcaaaggcgaat

SEQ ID NO: 28 - DNA, 16 bp, Artificial Sequence
  Primer sequence
cggcgagagccagttc

SEQ ID NO: 29 - DNA, 16 bp, Artificial Sequence
  Primer sequence
cggcgagagccagttt

SEQ ID NO: 30 - DNA, 16 bp, Artificial Sequence
  Primer sequence
cggcgagagccagatc

SEQ ID NO: 31 - DNA, 16 bp, Artificial Sequence
  Primer sequence
cggcgagagccagatt

SEQ ID NO: 32 - DNA, 21 bp, Artificial Sequence
  Primer sequence
ctgctggaggctgaagttctt

SEQ ID NO: 33 - DNA, 17 bp, Artificial Sequence
  Primer sequence
gcgaaccagaacggtag

SEQ ID NO: 34 - DNA, 18 bp, Artificial Sequence
  Primer sequence
cggaccacgacctttatc

SEQ ID NO: 35 - DNA, 21 bp, Artificial Sequence
  Primer sequence
acgacctttatcaaggaagga

SEQ ID NO: 36 - DNA, 20 bp, Artificial Sequence
  Primer sequence
aaratggataagccagaagc

SEQ ID NO: 37 - DNA, 19 bp, Artificial Sequence
  Primer sequence
tttcgctgaaacgtggayg

SEQ ID NO: 38 - DNA, 19 bp, Artificial Sequence
  Primer sequence
tttcgctgaaacgtggtyg

SEQ ID NO: 39 - DNA, 19 bp, Artificial Sequence
  Primer sequence
tttcgctgaaacgtggacg

SEQ ID NO: 40 - DNA, 19 bp, Artificial Sequence
  Primer sequence
tttcgctgaaacgtggtcg

SEQ ID NO: 41 - DNA, 23 bp, Artificial Sequence
  Primer sequence
tacgataaaaacaarctgttcgg

SEQ ID NO: 42 - DNA, 18 bp, Artificial Sequence
  Primer sequence
ccagagtgaccrccaatg

SEQ ID NO: 43 - DNA, 18 bp, Artificial Sequence
  Primer sequence
ccagagtgaccrccattg

SEQ ID NO: 44 - DNA, 18 bp, Artificial Sequence
  Primer sequence
ccagagtgacctccaatg

SEQ ID NO: 45 - DNA, 20 bp, Artificial Sequence
  Primer sequence
ccwttaytgtcgcagatccc

SEQ ID NO: 46 - DNA, 20 bp, Artificial Sequence
  Primer sequence
ctttcgcttccacgacttcg

SEQ ID NO: 47 - DNA, 20 bp, Artificial Sequence
  Primer sequence
ctttcgcttccacgactacg

SEQ ID NO: 48 - DNA, 18 bp, Artificial Sequence
  Primer sequence
ttcgcttccacgacatcg

SEQ ID NO: 49 - DNA, 18 bp, Artificial Sequence
  Primer sequence
ggcgtrgcragtaagccc

SEQ ID NO: 50 - DNA, 18 bp, Artificial Sequence
  Primer sequence
ggcgtrgcrcgtaagacc

SEQ ID NO: 51 - skipped

SEQ ID NO: 52 - DNA, 17 bp, Artificial Sequence
  Primer sequence
atgaaagttgcagtcct

SEQ ID NO: 53 - DNA, 16 bp, Artificial Sequence
  Primer sequence
gcgtggcggttgatct

SEQ ID NO: 54 - DNA, 22 bp, Artificial Sequence
  Primer sequence
gcttttttcagyacttctkgcg

SEQ ID NO: 55 - DNA, 22 bp, Artificial Sequence
  Primer sequence
cggcttttttcagyacttckgc

SEQ ID NO: 56 - DNA, 22 bp, Artificial Sequence
  Primer sequence
cggcttttttcagyacatckgc

SEQ ID NO: 57 - DNA, 17 bp, Artificial Sequence
  Primer sequence
atgtcgtacaacgagag

SEQ ID NO: 58 - DNA, 16 bp, Artificial Sequence
  Primer sequence
agatcaaccgccacgc

SEQ ID NO: 59 - DNA, 18 bp, Artificial Sequence
  Primer sequence
ggakatcagcacyacatc

SEQ ID NO: 60 - DNA, 21 bp, Artificial Sequence
  Primer sequence
atcttctacgaagtggccgtt

SEQ ID NO: 61 - DNA, 23 bp, Artificial Sequence
  Primer sequence
gcgatgatctggttaactagatc